(19) United States
(12) Patent Application Publication
(10) Pub. No.: US 2010/0197812 A1
(43) Pub. Date: Aug. 5, 2010

(54) Compositions and methods for enhancing the stability of foods, beverages, nutritional supplements and cosmetics

(75) Inventors: Roger Nahas, Kalamazoo, MI (US); Lucas Chadwick, Ann Arbor, MI (US); Donald Berdahl, Lawton, MI (US); Jill McKeague, Kalamazoo, MI (US); Peter Collins Vanalstyne, Paw Paw, MI (US); Anita E. Uhlir, Paw Paw, MI (US); Gregory S. Reynhout, Kalamazoo, MI (US); Thomas S. Jones, Kalamazoo, MI (US); James Barren, Kalamazoo, MI (US)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: KALAMAZOO HOLDINGS, INC.

(21) Appl. No.: 12/658,178

(22) Filed: Feb. 4, 2010

Related U.S. Application Data
(60) Provisional application No. 61/206,779, filed on Feb. 4, 2009.

(51) Int. Cl. A61K 47/46; A23L 1/28; A23L 3/34; A61K 8/97; A61Q 90/00; A61P 43/00

(52) U.S. Cl. 514/783; 426/655; 426/542; 426/532; 426/271

(57) ABSTRACT

The present invention provides metal-chelating or sequestering antioxidant compositions, derived from edible herbs, spices, fruits, vegetables and/or grains, useful for incorporating into foods, beverages, nutritional supplements and cosmetics for the purpose of enhancing the stability of said food, beverage or cosmetic. This invention provides processes for preparing these antioxidant, stability-enhancing compositions.

### FIELD OF THE INVENTION

The present invention generally relates to compositions and methods for enhancing the stability of foods, beverages, nutritional supplements and cosmetics. Furthermore, the present invention relates to processes for preparing metal chelating or sequestering antioxidant compositions with specific activities and solubility characteristics tailored to deliver the metal chelating and other antioxidant components to sites within foods, beverages, nutritional supplements or cosmetics where they may operate most effectively. The present invention further relates to foods, beverages, nutritional supplements and cosmetics treated with the inventive compositions. Finally, the invention pertains to such metal chelating compositions derived from edible herbs, spices, fruits, and/or vegetables, optionally comprising one or more non-chelating antioxidant component also derived from edible herbs, spices, fruits, vegetables and/or grains, and/or further optionally combined with one or more synthetic food grade antioxidants.

### BACKGROUND OF THE INVENTION

Many problems which affect the stability of foods, beverages, nutritional supplements and/or cosmetics may be solved, or at least kept under control, by adequate technological intervention. There is a continual need for enhancing the stability of foods, beverages, nutritional supplements and/or cosmetics. Enhanced stability can include flavor stability, color stability, textural stability and/or component stability (such as lipid, vitamin, carotenoid, protein or other constituent).

Substances that serve to protect foods from the deleterious effects of oxidation are commonly added to foods and are called antioxidants or stabilizers. These substances can be naturally or synthetically derived, although consumers generally prefer those materials from natural sources. The performance of a given antioxidant is dependent upon many things, including its chemical nature (stability, reactivity, functionality and the like) and its physical properties (volatility, solubility, polarity and the like). Antioxidant substances can have different modes of action, interfering with oxidation processes in a number of ways. Substances can function as antioxidants if they:
Disrupt the oxidation mechanism by reacting with free radical intermediates (radical scavengers).
React preferentially with oxygen, removing it from the environment of the substrate being stabilized (oxygen scavengers).
Absorb, and render less harmful, energy from incident radiation or energy from excited chemical species (quenchers).
Reduce and thereby regenerate oxidized antioxidants (AO regenerators).
Reduce peroxidic intermediates to non-radical products (secondary antioxidants).
Sequester and lessen the activity of metal initiators of oxidation (metal chelators).

In addition, in order to be effective in a complex food, beverage, nutritional supplement and/or cosmetic, antioxidant substances must possess the right solubility properties to allow them to migrate to the site in the substrate matrix where the oxidation or the initiation of oxidation is taking place.

Some of the most commonly used and most effective metal chelating additives in foods, beverages, cosmetics and nutritional supplements are derivatives of the synthetic compound ethylenediamine tetraacetic acid (EDTA). Based on the structure, EDTA is a very powerful metal chelator. EDTA is particularly useful in stabilizing oil and water-containing emulsion systems, such as mayonnaise, salad dressings, emulsified beverages, and the like. Since EDTA has many industrial applications, it has become widespread in the environment and is the most abundant man-made compound in many European surface waters. Although the isolated molecule does not present a risk of bioaccumulation, the ligand-metal complexes may significantly increase the bioavailability of extremely dangerous heavy metals. (Oveido and Rodŕiguez, 2003). Because of these concerns and the consumer preference for natural, as opposed to synthetic additives, there is a need to find a natural, preferably GRAS (Generally Recognized As Safe) replacement for this important and highly functional food additive.

In U.S. Pat. No. 6,123,945, now lapsed, Nakatsu and Yamasaki describe water soluble antioxidant compositions which were prepared by extracting defatted herb residues with hydrated alcohol. Examples of the method included the extraction of rosemary and clary sage roots, and it was suggested that water-soluble antioxidants could be prepared from mace, thyme, oregano, nutmeg, ginger, cinnamon, clove, basil, marjoram, mustard, savory, laurel, anise and the like. The water-soluble antioxidants were disclosed to be useful in fruit juices, processed meat foods, such as ham and sausage, processed aquatic foods, butter, margarine, mayonnaise, salad dressings, scented toiletries, soap, shampoo, detergent lotion, foundation, aromatic agents, hair styling material, and essential oils, such as lemon oil, lime oil, grapefruit oil, orange oil and the like. The water soluble antioxidants were not described as having chelating properties.

In U.S. Pat. No. 6,383,543 B1, Reznik describes water soluble antioxidants comprising sodium rosmarinate prepared by extracting tissue of plants of the Labiatae family with weakly acidic, neutral or alkaline aqueous extracts, optionally after extraction with water-immiscible organic solvents. Methods for isolation of a purified form of sodium rosmarinate are also described. The effect of the water-soluble antioxidants were demonstrated in model systems, including oil in water emulsions, emulsions containing beta-carotene, bulk vegetable oil, and essential oils. The water soluble antioxidants were not described as having chelating properties.

In U.S. Pat. No. 4,380,506, Kimura and Kanamori describe further extracting residues of herbs which have been previously extracted to form an oleoresin, producing a preservative with anti-oxidative and antimicrobial activity. This extraction of spent material was done with a mixture of polar and non-polar solvents. The polar solvents include ethyl ether, ethylene chloride, dioxane, acetone, ethanol, hydrous ethanol, methanol, ethyl acetate, propylene glycol and glycerin. Non-polar solvents include n-hexane, petroleum ether, ligroin, cyclohexane, carbon tetrachloride, chloroform, dichloromethane, 1,2-dichloroethane, toluene and benzene. The preservative prepared was described as being suitable for adding to oily and fatty foods, oil and fat containing foods, other foods, cosmetics and medicines.

### OBJECTS OF THE INVENTION

It is an object of the present invention to provide compositions for stabilizing foods, beverages, nutritional supplements and cosmetics, using as one component, spent herb, spice, vegetable and/or fruit extracts with metal chelating properties. It is a further object of the present invention to provide methods for enhancing the stability of foods, beverages, nutritional supplements and/or cosmetics by incorporating into them, effective amounts of metal chelating antioxidant compositions derived from edible herbs, spices, fruits, and/or vegetables, optionally comprising one or more non-chelating antioxidant component also derived from edible herbs, spices, fruits, vegetables and/or grains, and/or further optionally combined with one or more synthetic food grade antioxidants. Other objects, features and advantages of the present invention will become apparent as one reads carefully through the descriptive examples, which examples are not in any way limiting.

### BRIEF SUMMARY OF THE INVENTION

What we therefore believe to be comprised by our invention may be summarized inter alia in the following words:

Compositions with high metal chelating activity and utility as antioxidants in food, beverages, nutritional supplements and cosmetics can be prepared by extracting vegetable matter with polar solvents, which polar solvents include water, methanol, ethanol, isopropyl alcohol, butanol, or mixtures of one or more of these, with the proviso that the vegetable matter had been previously been depleted of the majority of its oil-soluble constituents by extraction with relatively non-polar solvents, including hexane, acetone, liquid carbon dioxide, supercritical carbon dioxide, tetrafluoroethane, petroleum ether, methyl ethyl ketone, ethyl acetate or mixtures, one or more, thereof. The vegetable matter comprises edible herbs, spices, fruits and vegetables. The vegetable matter can optionally be subjected to steam distillation to remove volatile oils at some point prior to the polar solvent extraction step.

The present invention relates to and relies upon the surprising solubility and metal chelating characteristics of antioxidant compositions derived from herbs, spices, fruits, and/or vegetables, which allow highly effective stabilizing or antioxidative compositions to be created from specific extracts of herbs, spices, fruits and/or vegetables, or their combinations.

The present invention further relates to highly effective antioxidant compositions made up of combinations of metal chelating elements derived from herbs, spices, fruits and/or vegetables, optionally, together with radical scavengers, oxygen scavengers, secondary antioxidants, quenchers and/or antioxidant regenerators derived from natural and/or synthetic sources.

The present invention thus provides methods for stabilizing foods, beverages, cosmetics and/or nutritional supplements by the application of herb, spice, fruit and/or vegetable-derived metal chelating compositions, optionally containing additional natural and/or synthetic antioxidants to the said food, beverage, cosmetic and or nutritional supplement, in an amount sufficient to have a measurable stabilizing effect.

The present invention further provides stabilized foods, beverages, cosmetics and/or nutritional supplements in the form of comprising a food, beverage, cosmetic and/or nutritional supplement together with a stabilizing composition consisting of metal chelating elements derived from herbs, spices, fruits, and/or vegetables, and, optionally, may be combined with synthetic and/or natural antioxidants of the radical scavenger, oxygen scavenger, secondary antioxidant, quencher and/or antioxidant regenerator types.

A plant extract composition derived from previously extracted edible herbs, spices, fruits and/or vegetable matter which have been depleted of the majority of oil-soluble constituents, wherein the plant extract composition exhibits metal chelating activity, such a

plant extract composition which exhibits anti-microbial and/or antimycotic activity in food compositions into which it is incorporated, such a

plant extract composition comprising a combination of metal chelating elements derived from previously extracted edible herbs, spices, fruits and/or vegetables, such a

plant extract composition comprising combinations of metal chelating elements derived from edible herbs, spices, fruits and/or vegetables, further comprising radical scavengers, oxygen scavengers, secondary antioxidants, quenchers and/or antioxidant regenerators derived from natural and/or synthetic sources, such a

stabilized food, beverage, cosmetic and/or nutritional supplement comprising metal chelating elements derived from herbs, spices, fruits, and/or vegetables, and, optionally, in combination with synthetic and/or natural antioxidants of the radical scavenger, oxygen scavenger, secondary antioxidant, quencher and/or antioxidant regenerator types, such a

method for stabilizing foods, beverages, cosmetics and/or nutritional supplements comprising incorporating a herb, spice, fruit and/or vegetable-derived metal chelating composition into the food, beverage, cosmetic and or nutritional supplement in an amount sufficient to have a measurable stabilizing effect, such a

method further comprising incorporating natural and/or synthetic antioxidants into the food, beverage, cosmetic and or nutritional supplement, such a

method for stabilizing the fresh flavor and preventing the formation of off-flavors in mayonnaise, salad dressings and other oil-in-water emulsion-based food systems by treating the mayonnaise, salad dressings and other oil-in-water emulsion-based food systems with an effective amount of a metal chelating antioxidant composition extracted from spice, herb, fruit and/or vegetable matter using a polar solvent or polar solvent mixture, said spice, herb, fruit and/or vegetable matter having previously been defatted by extraction with a relatively non-polar solvent or solvent mixture, said antioxidant composition, optionally comprising one or more non-chelating antioxidant components also derived from edible herbs, spices, fruits, vegetables and/or grains, and/or further optionally combined with one or more synthetic food grade antioxidants, in a manner that does not cause an objectionable off-color in the mayonnaise, salad dressing or other oil-in-water emulsion-based food, such a

method for stabilizing the fresh flavor and color and preventing the formation of off-flavors and off-colors in cured meats comprising incorporating into these food compositions an effective amount of a metal chelating antioxidant composition extracted from spice, herb, fruit and/or vegetable matter using a polar solvent or polar solvent mixture, said spice, herb, fruit and/or vegetable matter having previously been defatted by extraction with a relatively non-polar solvent or solvent mixture, said antioxidant composition optionally comprising one or more non-chelating antioxidant components also derived from edible herbs, spices, fruits, vegetables and/or grains, and/or further optionally combined with one or more synthetic food grade antioxidants, such a

method wherein the cured meat is selected from ham, bacon, salt pork, sausage, kippered herring, beef jerky, salami, summer sausage, cold cuts, bologna, pastrami, pepperoni, corned beef, roast beef, hot dogs, dried beef, bratwurst, polish sausage, barbequed pork, pork loin, beef brisket, salmon, liverwurst, pork char sui, prosciutto, culatello, lomo, coppa, bresaola, lardo, guanciale, mocetta, and qadid, such a

method for stabilizing the fresh flavor and preventing the formation of off-flavors in frying oils and in the foods fried in that oil by treating the frying oil prior to or during the frying operation with an effective amount of a metal chelating antioxidant composition extracted from spice, herb, fruit and/or vegetable matter using a polar solvent or polar solvent mixture, said spice, herb, fruit and/or vegetable matter having previously been defatted by extraction with a relatively non-polar solvent or solvent mixture, said antioxidant composition, optionally comprising one or more non-chelating antioxidant components derived from edible herbs, spices, fruits, vegetables and/or grains, and/or further optionally combined with one or more synthetic food grade antioxidants, such a

method for slowing the rate of oxidation, stabilizing the fresh flavor and preventing the formation of off-flavors in extruded human and animal foods comprising incorporating an effective amount of a metal chelating antioxidant composition extracted from spice, herb, fruit and/or vegetable matter using a polar solvent or polar solvent mixture, said spice, herb, fruit and/or vegetable matter having previously been defatted by extraction with a relatively non-polar solvent or solvent mixture, said antioxidant composition, optionally comprising one or more non-chelating antioxidant components derived from edible herbs, spices, fruits, vegetables and/or grains, and/or further optionally combined with one or more synthetic food grade antioxidants, such a

a method for slowing the rate of oxidation, stabilizing the fresh flavor and preventing the formation of off-flavors in fats and oils comprising polyunsaturated lipids comprising treating the fats and oils with an effective amount of a metal chelating antioxidant composition extracted from spice, herb, fruit and/or vegetable matter using a polar solvent or polar solvent mixture, said spice, herb, fruit and/or vegetable matter having previously been defatted by extraction with a relatively non-polar solvent or solvent mixture, said antioxidant composition, optionally comprising one or more non-chelating antioxidant components derived from edible herbs, spices, fruits, vegetables and/or grains, and/or further optionally combined with one or more synthetic food grade antioxidants, such a

method of slowing or preventing the growth of microorganisms in a food composition comprising incorporating an effective amount of a metal chelating antioxidant composition extracted from spice, herb, fruit and/or vegetable matter using a polar solvent or polar solvent mixture, said spice, herb, fruit and/or vegetable matter having previously been defatted by extraction with a relatively non-polar solvent or solvent mixture.

### DETAILED DESCRIPTION OF THE INVENTION

The present invention provides metal-chelating or sequestering antioxidant compositions, derived from edible herbs, spices, fruits, vegetables and/or grains, useful for incorporating into foods, beverages, nutritional supplements and cosmetics for the purpose of enhancing the stability of said food, beverage or cosmetic. This invention provides processes for preparing these antioxidant, stability-enhancing compositions.

We have found that antioxidative, natural metal chelating compositions useful for stabilizing foods, cosmetics, beverages and nutritional supplements can be prepared from certain spices, herbs, fruits and/or vegetables. The spices, herbs, fruits and vegetables that serve as sources of these antioxidants include, allspice, anise, star anise, caper, caraway, cardamom, capsicum pepper, cinnamon, clove, coriander, cumin, curry, dill, fennel, ginger, mace, nutmeg, marjoram, mustard, paprika, black pepper, white pepper, saffron, sage tarragon, thyme, turmeric, rosemary, galangal, balm, basil, grains of paradise, bay, basil, celery, licorice, mint, mistletoe, parsley, peppermint, valerian, vanilla, carrot, potato, tomato and the like. The antioxidant substances extracted from these spices, herbs, vegetables and/or fruits can be combined to form more complex antioxidant compositions. The antioxidant compositions can be obtained by processes known to those skilled in the art.

This invention provides processes for preparing these antioxidant, stability-enhancing compositions. One method of obtaining the metal chelating compositions is by a two-step extraction process. In the first step, spices, herbs, fruits and/or vegetables are extracted with a solvent such as hexane, pentane, butane, liquefied propane, acetone, methanol, ethanol, ethyl acetate, methyl ethyl ketone, super critical carbon dioxide, tetrafluoroethane, methylene chloride or any combination thereof. This generates an extract solution or suspension and a residue. In the second step, the residue is extracted with water or a water/polar solvent combination to generate a second extract solution or suspension and a second residue. The polar solvents used can be water, methanol, ethanol, isopropyl alcohol, butanol, propylene glycol, glycerin, acetone, methyl ethyl ketone, tetrahydrofuran or any combination thereof. The second extract solution or suspension, if made with water or an edible solvent, may be suitable to be added directly to certain foods, cosmetics, nutritional supplements or beverages at some point in their manufacture, providing a metal sequestering or chelating function.

Alternatively, the metal chelating portion of the second extract solution or suspension may be separated from the solvent portion by a number of means known in the art, providing a metal chelating composition. The solvent can be distilled away from the metal chelating portion at atmospheric pressure or reduced pressure.

Optionally, the metal chelating portion of the second extract solution or suspension can be further refined by a number of processes well known in the art, such as partitioning, precipitation, crystallization or recrystallization, to name just a few.

Alternatively, the second extract solution or suspension may be applied to a solid support system that selectively binds to the metal chelating portion of the extract solution or suspension. The solid support can be separated from the solution or suspension and the metal chelating portion freed from the solid support by a number of desorptive processes known in the art. Optionally, the solid support containing the adsorbed metal chelating portion can be treated with various agents for the purpose of removing or reducing the levels of unwanted substances prior to the desorption step. These unwanted substances might contribute to undesirable colors, flavors or other attributes that would be detrimental in the final product.

Alternatively, the extract solution or suspension can be spray dried using processes well known in the art, providing the metal chelating composition in solid form. Optionally, substances useful for aiding in the spray drying process and/or for, controlling the characteristics of the solid obtained can be added.

Another method for obtaining metal chelating compositions is by a multiple-step extraction process. This process is particularly useful in that the metal chelating composition is obtained in the form of a series of fractions with differing color, flavor, polarity, solubility and metal chelating attributes. These fractions can be used separately or recombined in various and selective ways to generate new compositions with color, flavor, polarity, solubility and metal chelating properties tailored for specific applications.

In the first step, spices, herbs, fruits and/or vegetables are extracted with a solvent such as hexane, pentane, butane, liquefied propane, acetone, methanol, ethanol, ethyl acetate, methyl ethyl ketone, super critical carbon dioxide, tetrafluoroethane, methylene chloride or any combination thereof. This generates an extract solution or suspension and a residue. In the second step, the residue is extracted with water or a water/polar solvent combination to generate a second extract solution or suspension and a second residue. The polar solvents used can be water, methanol, ethanol, isopropyl alcohol, butanol, propylene glycol, glycerin, acetone, methyl ethyl ketone, tetrahydrofuran or any combination thereof. In the third step, the second residue is further extracted with water or a water/polar solvent combination to generate a third extract solution or suspension and a third residue. The polar solvents used can be water, methanol, ethanol, isopropyl alcohol, butanol, propylene glycol, glycerin, acetone, methyl ethyl ketone, tetrahydrofuran or any combination thereof. In a fourth step, the third residue is further extracted with water or a water/polar solvent combination to generate a fourth extract solution or suspension and a fourth residue. The polar solvents used can be water, methanol, ethanol, isopropyl alcohol, butanol, propylene glycol, glycerin, acetone, methyl ethyl ketone, tetrahydrofuran or any combination thereof. This process can continue through a fifth, sixth or seventh step, and can in theory continue indefinitely. Practical considerations such as cost, the presence of metal chelating constituents in the nth extract, and their utility, will govern the extent to which the extraction process is performed. Different solvents or solvent mixtures can be used in each of these extraction steps, allowing for the formation metal chelating compositions with varying attributes. Each of the extract solutions or suspensions produced may be used directly or further processed as described above. The extract solution or suspension, if made with water or an edible solvent, may be suitable to be added directly to certain foods, cosmetics, nutritional supplements or beverages at some point in their manufacture, providing a metal sequestering or chelating function. Alternatively, the metal chelating portion of the inventive extract solution or suspension can be separated from the solvent portion by a number of means known in the art, providing a metal chelating composition. The solvent can be distilled away from the metal chelating portion at atmospheric pressure or reduced pressure. Optionally, the metal chelating portion of the extract solution or suspension can be further refined by a number of processes well known in the art, such as partitioning, precipitation, crystallization or recrystallization, to name just a few. Alternatively, the solution or suspension in question may be applied to a solid that selectively binds to the metal chelating portion of the extract solution or suspension. The solid can be separated from the solution or suspension and the metal chelating portion freed from the solid by a number of desorptive processes known in the art. Optionally, the solid containing the adsorbed metal chelating portion can be treated with various agents for the purpose of removing or reducing the levels of unwanted substances prior to the desorption step. These unwanted substances might contribute to undesirable colors, flavors or other attributes that would be detrimental in the final product. Alternatively, the inventive extract solution or suspension can be spray dried using processes well known in the art, providing the metal chelating composition in solid form. Optionally, substances useful for aiding in the spray drying process and/or for controlling the characteristics of the solid obtained can be added. In some cases it may be beneficial to process each solution or suspension fraction separately. In other cases, it may be beneficial to combine certain solution or suspension fractions together and process the mixture into its final form.

In both of the cases described above, the extractions can be performed as a batch process or as a continuous process, using equipment suitable for each. The input spice, herb, fruit or vegetable matter can be in the form of fresh or dehydrated material. The extractions are generally more efficient when the biomass to be extracted is in a form with reduced particle size, i.e., ground, comminuted, chopped, shredded or the like.

Another method for obtaining metal chelating compositions involves subjecting the spice, herb, fruit or vegetable biomass to a steam distillation process whereby certain volatile materials are removed. This produces a water distillate fraction containing the materials volatile with steam, and a pot residue, consisting of the residual water in the pot together with the biomass residue. The pot residue can be filtered, generating a solid residue and an aqueous fraction. The aqueous fraction may be suitable to be added directly to certain foods, cosmetics, nutritional supplements or beverages at some point in their manufacture, providing a metal sequestering or chelating function. Alternatively, the metal chelating portion of the aqueous fraction can be separated from the water by a number of means known in the art, providing a metal chelating composition. The water can be distilled away from the metal chelating portion at atmospheric pressure or reduced pressure. Optionally, the metal chelating portion of the water solution or suspension can be further refined by a number of processes well known in the art, such as partitioning, precipitation, crystallization or recrystallization, to name just a few.

Alternatively, the aqueous fraction may be applied to a solid support system that selectively binds to the metal chelating portion of the extract solution or suspension. The solid support can be separated from the water and the metal chelating portion freed from the solid by a number of desorptive processes known in the art. Optionally, the solid support containing the adsorbed metal chelating portion can be treated with various agents for the purpose of removing or reducing the levels of unwanted substances prior to the desorption step. These unwanted substances might contribute to undesirable colors, flavors or other attributes that would be detrimental in the final product.

Alternatively, the aqueous fraction can be spray dried using processes well known in the art, providing the metal chelating composition in solid form. Optionally, substances useful for aiding in the spray drying process and/or for controlling the characteristics of the solid obtained can be added.

The biomass residue may be extracted with water or a water/polar solvent combination to generate a second extract solution or suspension and a second residue. The polar solvents used can be water, methanol, ethanol, isopropyl alcohol, butanol, propylene glycol, glycerin, acetone, methyl ethyl ketone, tetrahydrofuran or any combination thereof. The second residue may be further extracted with water or a water/polar solvent combination to generate a third extract solution or suspension and a third residue. The polar solvents used can be water, methanol, ethanol, isopropyl alcohol, butanol, propylene glycol, glycerin, acetone, methyl ethyl ketone, tetrahydrofuran or any combination thereof. The third residue may be further extracted with water or a water/polar solvent combination to generate a fourth extract solution or suspension and a fourth residue. The polar solvents used can be water, methanol, ethanol, isopropyl alcohol, butanol, propylene glycol, glycerin, acetone, methyl ethyl ketone, tetrahydrofuran or any combination thereof. This process can continue through a fifth, sixth or seventh step, and can in theory continue indefinitely. Practical considerations such as cost, the presence of metal chelating constituents in the nth extract, and their utility, will govern the extent to which the extraction process is continued. Different solvents or solvent mixtures can be used in each of these extraction steps, allowing for the formation metal chelating compositions with varying attributes. Each of the extract solutions or suspensions produced may be used directly or further processed as described above. Namely, the extract solution or suspension in question, if made with water or an edible solvent may be suitable to be added directly to certain foods, cosmetics, nutritional supplements or beverages at some point in their manufacture, providing a metal sequestering or chelating function. Alternatively, the metal chelating portion of the extract solution or suspension in question can be separated from the solvent portion by a number of means known in the art, providing a metal chelating composition. The solvent can be distilled away from the metal chelating portion at atmospheric pressure or reduced pressure. Optionally, the metal chelating portion of the solution or suspension can be further refined by a number of processes well known in the art, such as partitioning, precipitation, crystallization or recrystallization, to name just a few.

Alternatively, the solution or suspension in question may be applied to a solid support system that selectively binds to the metal chelating portion of the extract solution or suspension. The solid support can be separated from the solution or suspension and the metal chelating portion freed from the solid by a number of desorptive processes known in the art. Optionally, the solid support containing the adsorbed metal chelating portion can be treated with various agents for the purpose of removing or reducing the levels of unwanted substances prior to the desorption step. These unwanted substances might contribute to undesirable colors, flavors or other attributes that would be detrimental in the final product.

Alternatively, the extract solution or suspension in question can be spray dried using processes well known in the art, providing the metal chelating composition in solid form. Optionally, substances useful for aiding in the spray drying process and/or for controlling the characteristics of the solid obtained can be added. In some cases it may be beneficial to process each solution or suspension fraction separately. In other cases, it may be beneficial to combine certain solution or suspension fractions together and then process the mixture into its final form.

The residues left over from the steam distillation production of clove, allspice, cinnamon, rosemary, oregano, sage, ginger, mace, nutmeg, cassia, marjoram, thyme, tarragon, spearmint, peppermint, anise, basil, and black pepper volatile oils are good sources of the chelator compositions of the present invention, and utilize a waste product that would otherwise have very little value. These residues from steam distillation may need to be extracted to remove lipidic materials prior to being re-extracted with a solvent of higher polarity to remove the chelator fraction.

A surprising feature of these methods of preparation, and one allowing great utility in the formulation of highly effective antioxidative compositions, is that the metal chelating compositions obtained from different spices, herbs, fruits and/or vegetables vary in their polarity and solubility characteristics.

Neither this feature of these compositions, nor the magnitude of the polarity and solubility differences has been previously recognized. One can combine these materials derived from different spices, herbs, fruits and/or vegetables to provide mixtures containing blended active ingredients with an optimized range of solubility and polarity characteristics capable of providing an improved antioxidative effect for a given food, beverage, cosmetic or nutritional supplement application.

While the underlying cause of the highly effective antioxidative performance characteristics of these compositions in a food, beverage, cosmetic or nutritional supplement is not completely understood, it may be due in part to metal chelating compounds with suitable solubility and polarity characteristics being efficiently transported to sites where they can act to maximum effect in these complex, multiphase systems. It is surprising and completely unpredicted that, for example, the water or polar solvent extraction of carrot residue gives a metal chelating composition that is exceedingly hydrophilic, whereas the use of the same extraction solvents and steps in the extraction of clove or allspice gives a metal chelating composition that is more readily dispersible in lipids. Other spices, herbs, fruits and/or vegetables that have been examined provide chelating compositions intermediate in polarity and solubility between that of carrot or allspice and clove. As noted above, the solubility and polarity characteristics of the metal chelating compositions can be further manipulated by the choice of solvents used in the single or multiple extraction steps of the spent herb, spice, fruit and/or vegetable biomass, providing an even greater mechanism for obtaining compositions with the desired solubility and polarity characteristics.

While the causes of the antioxidant effect of the compositions of the present invention are only incompletely understood, metal chelating effects of varying degrees have been identified in all the compositions described, as demonstrated by the results of the Ferrozine Assay as shown in Table 1, and is thought to be most responsible for the stabilizing properties of the extracts. A radical scavenging effect has also been detected for some of the extract compositions that have been prepared. This is not entirely unexpected, since phenolic compounds of various kinds are likely constituents of the extracts and phenolic compounds are known to possess radical scavenging capabilities due to their well-known ability to donate hydrogen atoms to radicals. The antioxidative effects of these compositions have been demonstrated using model screening systems such as the Ferrozine Assay, which measures the ability of a compound to bind to ferrous iron (Fe+2), the DPPH test, which measures the radical scavenging ability of compositions by measuring the ability to bleach the diphenylpicryl hydrazyl radical, and using carotenoid bleaching assays. The antioxidant effects of these extracts, and their combinations with other natural and/or synthetic antioxidants, have also been evaluated in simple food models and in actual food, beverage, nutritional supplement and/or cosmetic applications.

We have, surprisingly, found that many of the inventive antioxidant, metal chelating compositions have an anti-microbial effect in the foods into which they are incorporated, even though their volatile or essential oil content is extremely low. The inventive antioxidant compositions, in part in consequence of their method of preparation, are very low in volatile constituents, such as essential oils. The well known antimicrobial effect of herbs and spices has been largely attributed to the action of their volatile (essential) oil constituents.

An embodiment of the instant invention relates to a combination of chelating compositions derived from herbs, spices, fruits and/or vegetables with other natural antioxidants, including, but not limited to, tocopherols, tocotrienols, ascorbic acid, ascorbates, natural gallates, catechins, epigallocatechin gallate, grape seed extract, olive leaf extract, resveratrol, carbazoles, erythorbic acid, erythorbates, carnosol, carnosic acid, rosmarinic acid, rosmanol, xanthohumol, rosemary extract, sage extract, oregano extract, and other spice and herb extracts wherein the majority of the antioxidant activity is due to the presence of radical scavenging agents. By carefully blending materials, it is possible to create antioxidant formulations that contain a complete contingent of oil soluble or dispersible radical scavenging agents, water soluble or dispersible radical scavenging agents, oil soluble or dispersible chelating agents, and water soluble or dispersible chelating agents, or any combination thereof. In this way the antioxidative elements of the composition can be more effectively delivered to the various polar, non-polar phases and intermediate polarity phases found in multiphase foods, cosmetics, beverages or nutritional supplements.

Another embodiment of the present invention involves the combination of chelating compositions derived from herbs, spices, fruits and/or vegetables with synthetic antioxidants such as propyl gallate, BHA, BHT, ethoxyquin, TROLOX®, TBHQ, ascorbyl palmitate, and EDTA. While these compositions are not as preferred as their all-natural counterparts, they are contemplated as part of the present invention in a manner as described in the previous paragraph.

An embodiment of the present invention involves the use of the metal chelating compositions, alone, or in combination with other natural or synthetic antioxidants in the stabilization of foods, beverages, cosmetics and nutritional supplements.

An embodiment of the present invention includes foods, beverages, cosmetics, and nutritional supplements treated with the metal chelating compositions, alone, or in combination with other natural or synthetic antioxidants.

The instant spent extract metal chelating compositions may be added directly to foods, where their solubility characteristics permit. They can be dissolved in a carrier, such as propylene glycol, glycerin, phospholipids, food grade surfactants, ethanol, benzyl alcohol, and the like, and then added to foods. They can be dispersed onto solid carriers, such as salt, flour, sugars, maltodextrin, silica (such as CAB-O-SIL®), cyclodextrins, starches, gelatins, lactose, whey powders, proteins, and the like and then added to foods.

The instant metal chelating compositions can be added directly to cosmetics including, but not limited to, lip balm, lip gloss, lipstick, lip stains, lip tint, blush, bronzers & highlighters, concealers & neutralizers, foundations, foundation primer, glimmers & shimmers, powders, eye shadow, eye color, eye liner, mascara, nail polish, nail treatments-strengtheners, make-up, body creams, moisturizers, suntan preparations, sunless tan formulations, body butter, body scrubs, make-up remover, shampoos, conditioners, dandruff control formulations, anti-frizz formulations, straightening formulations, volumizing formulations, styling aids, hairsprays, hair gels, hair colors and tinting formulations, anti-aging creams, body gels, essential oils, creams, cleansers, and soaps.

The instant metal chelating compositions may be added directly to beverages including, but not limited to, beer, wine, teas, herbal tea, coffee, cappuccino, espresso, café au lait, frappes, lattes, soft drinks (carbonated and still), fruit juices, vegetable juices, milks, lemonades, punches, chocolates, ciders, chai, dairy beverages, smoothies, energy drinks, alcoholic beverages, brandies, gin, vodka, fortified waters, flavored waters, whiskey, distilled spirits, bourbon, and malt liquor.

The instant metal chelating compositions may be added directly to human and animal foods including, but not limited to, meat (wild and domestic; fresh and cured, processed and unprocessed, dried, canned), poultry, fish, vegetable protein, dairy products (milk, cheese, yogurt, ice cream), ground spices, vegetables, pickles, mayonnaise, sauces (pasta sauces, tomato-based sauces), salad dressings, dried fruits, nuts, potato flakes, soups, baked goods (breads, pastries, pie crusts, rolls, cookies, crackers, cakes, pies, bagels), vegetable oils, frying oil, fried foods (potato chips, corn chips), prepared cereals (breakfast cereals), cereal grain meals, condiments (ketchup, mustard, cocktail sauce, candies, confectionery, chocolates, and baby foods

Animal foods, for example, include but are not limited to, extruded pet food, kibbles, dry pet food, semi-dry pet food, and wet pet food.

The instant metal chelating compositions may be added to nutritional supplements, including, but not limited to, eye health supplements, vitamins, nutrition boosters, carotenoid supplements, protein supplements, energy bars, nutritional bars, algal oils, fish oils, and oils containing polyunsaturated fatty acids.

Embodiments of the instant invention include: prevention of color loss in fresh meat, stabilization of seasoning flavor in spice blends and seasoned foodstuffs, prevention of rancidity in baked goods, prevention of rancid flavor development in snack foods, stabilization of foods and nutritional supplements against nutrient and vitamin loss through oxidative processes, stabilization of extruded human and pet foods, stabilization of phospholipids, lard, butter, margarine, spreads, stabilization of fats and oils in the rendering process, stabilization of canned tomato sauce and paste, canned fruits and vegetables, stabilization of pickle flavor and color, stabilization of potato flakes, stabilization of breakfast cereals, stabilization of essential oils (whole) or essential oil components in foods and beverages (citrus, orange oil, mint oils etc.), stabilization of peanut butter, almonds, walnuts, hazel nuts, peanuts, macadamia nuts, brazil nuts, stabilization of rice bran oil, stabilization of fish and poultry (fresh and cooked), stabilization of corn masa, fried or baked corn chips, stabilization of chocolate (light and dark), stabilization of oleoresins, and stabilization of soft drinks containing ascorbic acid and sodium benzoate, to mitigate formation of benzene, prevention of color loss in cosmetics, prevention of oxidative changes in cosmetics leading to off aroma development, discoloration, and loss of functional ingredients.

The spent plant extract compositions of the present invention have metal chelating properties. For the purpose of this invention, the term metal ions may be defined as those metal ions that promote or initiate lipid or other oxidation processes, including, but not limited to Fe+2, F2+3, Cu+1, Cu+2, Ni+2. One method for measuring the metal chelating strength of a substance is the so-called Ferrozine Assay. Ferrozine (3-(2-pyridyl)-5,6-diphenyl-1,2,4-triazine-4′-4″-disulfonic acid, sodium salt) is commonly used to assess the potential of materials to chelate Fe(II). Ferrozine forms a colored complex with Fe(II) with a maximum absorbance at 562 nm [Carter, 1971]. The potency of extracts or pure compounds to bind ferrous ions is assessed by their competition with ferrozine, resulting in a decrease in the formation of the colored complex. The degree of color fading is assessed by measuring the absorbance at 562 nm and correlated to the strength by which the chelator binds to the metal.

The spent plant extracts described in the present invention have metal chelating properties, but some also serve as radical scavengers. The DPPH (2,2-diphenyl-1-picrylhydrazyl) radical is commonly used to test the radical scavenging potential of antioxidants. DPPH has a characteristic purple color due to its absorbance at 515-520 nm. When DPPH pairs off its odd electron in the presence of a hydrogen-donating free radical scavenger, it loses its color. The efficacy of the antioxidant can be determined from the degree of DPPH color fading. The DPPH method is fast, reproducible and does not require special equipment. (Koleva et al., 2002).

Model systems that are simpler representations of foods, beverages, nutritional supplements and cosmetics can also be used to test the performance of antioxidant compositions. An assay was developed to predict the behavior of a metal chelator in a simple oil matrix using the Oxidative Stability Index method (OSI) at 90° C. with unfortified canola oil. Tests can be performed in the presence and absence of added metal salts. The data has shown that although a radical scavenger like TROLOX® improves the oxidative stability remarkably in absence of metals, it is totally ineffective when metals are added [TROLOX® is a registered trademark of Hoffmann-LaRoche]. A metal chelator like EDTA can improve the oxidative stability in presence of metal ions, but does not extend the stability of the oil in the absence of metal ions. By comparing the behavior of an oil/extract combination in the absence of metal ions to the behavior in the presence of metal ions, the metal chelating potential of the extract can be determined in an oil matrix.

Measuring the rate of fading of carotenoid pigments can also be used as a test of antioxidant performance. Carotenoid pigments possess a high number of conjugated double bonds resulting in high reactivity towards free radicals, oxidation, subsequent degradation and loss of color. One of the tests employed to measure the antioxidant strength of the present inventive compositions involved measuring the rate at which carotenoids in paprika oleoresin faded in treated and untreated samples.

Food systems than contain polyunsaturated fats are subject to lipid oxidation leading to food quality deterioration and formation of off-flavors. Oxidation can be monitored by measuring the primary oxidation products (hydroperoxides) as well as secondary oxidation products (aldehydes and ketones). The conjugated diene hydroperoxide test is a spectroscopic method that allows the assessment of the oxidative stability of a bulk oil system or oil and water emulsion system and the efficacy of antioxidant treatments. Due to lack of conjugated dienoic systems, un-oxidized lipids do not usually absorb UV light at wavelengths higher than ˜210 nm. However, as conjugated hydroperoxides form as a result of oxidation, the absorptivity at 234 nm increases because the conjugated double bonds in these primary oxidation products absorb UV light at this wavelength. Most of the emulsion models established to mimic food systems and used as a matrix to test the performance of various antioxidants consist of oil-in-water emulsions (O/W). Foods containing O/W emulsions include mayonnaise, milk, cream, etc.

In butter and margarine, oil (the continuous phase) surrounds droplets of water (the discontinuous phase). These are water-in-oil emulsions (W/O). There are few if any water-in-oil emulsion models reported. A 20% W/O emulsion model was created using unfortified canola oil (80%), deionized water (20%), Atmos 300 K (mixture of mono- and diglycerides) or glycerol monooleate (2%) as an emulsifier to facilitate the dispersion of the water in the oil by reducing the surface tension in water, and Polyoxyethylene-(20)-sorbitan monolaurate (0.2%) to reduce surface tension in water.

Naturally-derived antioxidants are used with good effect as stabilizers in many food, beverage, nutritional supplements and cosmetics products. There are many products and ingredients, however, that are highly oxidatively unstable and for which the current state of the art antioxidants are insufficient to provide the degree of increased oxidative stability required or desired. It is an object of this invention to provide methods and compositions to improve the oxidative stability of products that are difficult to stabilize with known antioxidants.

### Mayonnaise

Much of the commercial mayonnaise sold around the world is stabilized with derivatives of the synthetic antioxidant, EDTA. EDTA is a very powerful chelating agent and is very effective in preserving the flavor of mayonnaise in storage. In Germany, EDTA is prohibited in mayonnaise. Absent the ability to use this highly effective stabilizer, German mayonnaise with sufficient shelf life must be manufactured with oils that are inherently more stable than the oils often used in mayonnaise in other countries, namely oils that are relatively more saturated. The use of more saturated fats runs counter to the desire to include more unsaturated fats in the diet, and there is a need to make and sell mayonnaise in Germany that incorporates more highly unsaturated and less inherently stable oils. Indeed, mayonnaise preparations containing highly unsaturated fish and algal-derived oils are desired. The current stabilizing agents allowed by German regulations are not sufficiently effective to stabilize mayonnaise made with higher levels of unsaturated oils. One purpose of this invention is to provide materials and methods to enhance the stabilization of mayonnaise and related oil-in-water emulsions such as salad dressings, dairy products, creamers and the like, beyond what is now practiced in the art.

Compositions of U.S. Pat. No. 6,123,945, (Nakatsu and Yamasaki) are antioxidants in some systems, but suffer from a complication which renders them not useful for stabilizing mayonnaise. We have found that the compositions of the '945 patent form a finely divided black precipitate when incorporated into mayonnaise which causes the mayonnaise to take on an undesirable grayish color. This phenomenon renders the compositions disclosed in U.S. Pat. No. 6,123,945 difficult, if not impossible, to use in this application. We have found that the propensity to form black precipitates in mayonnaise (and indeed, in bulk oils) is more pronounced for some of the '945 compositions, than for others. Clove, and, incidentally, allspice spent extracts form the highest levels of black, finely divided precipitates in mayonnaise.

We have discovered, surprisingly, that extracts of previously defatted vegetables, such as carrots or potato peelings, show potent metal chelating activity and the ability to stabilize mayonnaise and other oil-in-water emulsions, but do not form the precipitate that leads to the formation of the off-color.

### Cured Meats

Cured meats are subject to oxidation processes that result in the loss of desirable flavors, the formation of off-flavors, the loss of desirable cured meat pigment color, and the formation of undesirable colors, among other effects that cause a decrease in the shelf life of the product. Cured meats are also subject to the growth of bacteria, yeasts and molds that also shorten the shelf life of the product. An embodiment of the instant invention is to provide materials and methods to enhance the stabilization of cured meats beyond what is presently achieved with compositions known to those skilled in the art.

Fish, Algal, and Vegetable Oils with High Levels of Unsaturation.

Highly unsaturated oils are very susceptible to oxidation and they are, therefore, difficult to incorporate into food, beverage, nutritional supplement and cosmetic products. Unsaturated oil emulsions are particularly difficult to stabilize. An embodiment of the instant invention is to provide materials and methods to enhance the stabilization of fish, algal and vegetable oils with high levels of unsaturation, and the like, over that which is currently achieved with compositions described in the art.

### Frying Oil

The frying process subjects the frying oil and the article being fried to severe oxidative stress. Current state of the art antioxidants, both natural and synthetic, fail to provide the desired stabilizing effects. An embodiment of the instant invention is to provide materials and methods to improve the shelf life and quality of frying oils and of fried foods.

### Potted Meats

Meat products, including baby food preparations that are retorted in metal, glass or plastic containers, often suffer oxidative damage leading to off color formation particularly at the surface of the product. The development of off flavors can also occur during the retort process and in the period during which the product is stored prior to use. It is a further embodiment of this invention to provide materials and methods to stabilize potted meat products against oxidation resulting in flavor and color changes.

### Coffee and Coffee Concentrates

Coffee extracts or concentrates are replacing freshly brewed coffee in many retail settings. Freshly brewed coffee and coffee extracts or concentrates are susceptible to oxidative process leading to unwanted flavor changes. It is a further purpose of this invention to provide materials and methods to stabilize coffee and coffee extracts or concentrates against oxidatively induced flavor changes.

### Beer and Malt Beverages

Beer and other malt beverages undergo undesirable flavor changes as a result of oxidative processes during the brewing process and in storage. It is a further embodiment of this invention to provide materials and methods to increase the flavor stability and shelf life of beer and malt beverages.

### Natural and Artificial Coloring Agents

Many natural and synthetic coloring agents are oxidatively unstable. Color loss in meat, beverages, foods, cosmetics and in the coloring compositions, themselves, accompanies the oxidation of these materials. It is a further embodiment of this invention to provide materials and methods to stabilize natural and artificial coloring agents such as anthocyanins, carotenoids, xanthophylls, capsanthin, capsorubin, lutein, zeaxanthin, bixin, norbixin, astaxanthin, beta-cryptoxanthin, lycopene, beta-carotene, alpha carotene, FD&C colors, chlorophylls, myoglobin, oxymyoglobin, nitrosomyoglobin, carboxymyoglobin, carmine, carminic acid, turmeric extract, curcumin, annatto extract, paprika extract, carrot extract, tomato extract, algal extracts, beet extract, hyacinth extracts, gardenia extracts, spinach extracts and the like against oxidation resulting in color and flavor changes in foods, beverages, nutritional supplements, cosmetics or in the natural and artificial coloring agents, themselves.

### Irradiated Meats

Treatment of fresh meat, poultry and seafood with incident radiation as described in U.S. Pat. No. 6,099,897, herein incorporated by reference, induces unwanted oxidative changes in the color, flavor and storage stability in the final irradiated product. It is a further purpose of this invention to provide materials and methods to stabilize irradiated meat, poultry and fish products against oxidation resulting in flavor and color changes beyond what is now practiced in the art.

This invention provides a method for stabilizing the fresh flavor and preventing the formation of off-flavors in mayonnaise, salad dressings and other oil-in-water emulsion-based food systems by treating these materials at some stage in their production with an effective amount of a metal chelating antioxidant composition extracted from spice, herb, fruit and/or vegetable matter using a polar solvent or polar solvent mixture, said spice, herb, fruit and/or vegetable matter having previously been defatted by extraction with a relatively non-polar solvent or solvent mixture, said antioxidant composition, optionally comprising one or more non-chelating antioxidant components also derived from edible herbs, spices, fruits, vegetables and/or grains, and/or further optionally combined with one or more synthetic food grade antioxidants, in a manner that does not cause an objectionable off-color in the mayonnaise, salad dressing or other oil-in-water emulsion-based food.

This invention provides a method for stabilizing the fresh flavor and color and preventing the formation of off-flavors and off-colors in cured meats, including ham, bacon, salt pork, sausage, kippered herring, beef jerky, salami, summer sausage, cold cuts, bologna, pastrami, pepperoni, corned beef, roast beef, hot dogs, dried beef, bratwurst, polish sausage, barbequed pork, pork loin, beef brisket, salmon, liverwurst, pork char sui, prosciutto, culatello, lomo, coppa, bresaola, lardo, guanciale, mocetta, qadid, and the like, by incorporating into these food compositions at some stage in their production, an effective amount of a metal chelating antioxidant composition extracted from spice, herb, fruit and/or vegetable matter using a polar solvent or polar solvent mixture, said spice, herb, fruit and/or vegetable matter having previously been defatted by extraction with a relatively non-polar solvent or solvent mixture, said antioxidant composition optionally comprising one or more non-chelating antioxidant components also derived from edible herbs, spices, fruits, vegetables and/or grains, and/or further optionally combined with one or more synthetic food grade antioxidants. Many of the antioxidant, metal chelating compositions also surprisingly show anti-microbial activity in the food compositions into which they are incorporated, by slowing or preventing the growth of microorganisms.

This invention provides a method for stabilizing the fresh flavor and preventing the formation of off-flavors in frying oils and in the foods fried in that oil by treating the frying oil prior to or during the frying operation with an effective amount of a metal chelating antioxidant composition extracted from spice, herb, fruit and/or vegetable matter using a polar solvent or polar solvent mixture, said spice, herb, fruit and/or vegetable matter having previously been defatted by extraction with a relatively non-polar solvent or solvent mixture, said antioxidant composition, optionally comprising one or more non-chelating antioxidant components also derived from edible herbs, spices, fruits, vegetables and/or grains, and/or further optionally combined with one or more synthetic food grade antioxidants.

This invention provides a method for slowing the rate of oxidation, stabilizing the fresh flavor and preventing the formation of off-flavors in fats and oils comprising polyunsaturated lipids by treating these materials at some stage in their production or use with an effective amount of a metal chelating antioxidant composition extracted from spice, herb, fruit and/or vegetable matter using a polar solvent or polar solvent mixture, said spice, herb, fruit and/or vegetable matter having previously been defatted by extraction with a relatively non-polar solvent or solvent mixture, said antioxidant composition, optionally comprising one or more non-chelating antioxidant components also derived from edible herbs, spices, fruits, vegetables and/or grains, and/or further optionally combined with one or more synthetic food grade antioxidants.

This invention provides a method for slowing the rate of oxidation, stabilizing the fresh flavor and preventing the formation of off-flavors in extruded human and animal foods by incorporating into them at some stage in their production or use, an effective amount of a metal chelating antioxidant composition extracted from spice, herb, fruit and/or vegetable matter using a polar solvent or polar solvent mixture, said spice, herb, fruit and/or vegetable matter having previously been defatted by extraction with a relatively non-polar solvent or solvent mixture, said antioxidant composition, optionally comprising one or more non-chelating antioxidant components also derived from edible herbs, spices, fruits, vegetables and/or grains, and/or further optionally combined with one or more synthetic food grade antioxidants.

### EXAMPLES

HERBALOX® Seasoning is a registered trademark of KALSEC®, Inc. HERBALOX® Seasoning 41-19-34 comprises a water soluble rosemary extract containing rosmarinic acid. HERBALOX® Seasoning Type HT-O comprises an oil soluble rosemary extract, with a portion of the volatile oils removed, and comprising carnosic acid and carnosol. HERBALOX® Seasoning 41.088319 comprises an oil soluble rosemary extract, with a portion of the volatile oils removed, and comprising carnosic acid and carnosol. HERBALOX® Seasoning QS is a rosemary extract formulation containing carnosic and carnosol that is dispersible in oil and dispersible in water. HERBALOX® Seasoning Type 25 is an oil soluble rosemary extract containing carnosic acid and carnosol.

### Example 1

### Extraction and Screening of Spice, Herb, Fruit and Vegetable Matter

Separately, samples of spent rosemary (Rosmarinus officinalis), clove (Syzygium aromaticum), allspice (Pimenta dioica), oregano (Origanum vulgare), carrot (Daucus carota), black pepper, white pepper (Piper nigrum), paprika (Capsicum annuum), hop (Humulus lupulus), cassia (Cinnamomum aromaticum), nutmeg (Myristica s.), cardamom (Elettaria cardamomum), celery seed (Apium graveolens), coriander seed (Coriandrum sativum), anise (Pimpinella anisum), dill seed (Anethum graveolens), and chaff, the solid residue separated from fermented Tabasco chillies in the process of making Tobasco sauce, that had been previously extracted with organic solvents to remove oil-soluble antioxidant compounds, flavors and colors, were re-extracted as follows.

### First Extraction

The “spent” material (400-650 g.) was weighed into a 4 L plastic container and mixed with MeOH at a 3 L to 1 Kg ratio of solvent to plant material. The resulting slurry was then processed using an IKA-Werke Ultra Turrax T50 Basic high sheer mixer for 2 minutes. The slurry was subsequently vacuum-filtered on a Büchner filter through Whatman 1 filter paper. The extraction filtrates were concentrated initially under reduced pressure at 50° C. on a rotary evaporator, then transferred into a Savant Speed-Vac concentration system and dried overnight at 55° C. resulting in weight yields of Extract 1 varying between 0.2% and 8.26%.

### Second Extraction

The solid residue from the first extraction was re-extracted using a mixture of H₂O:MeOH (1:3, by volume), processed with the high sheer mixer for 2 minutes, and subsequently vacuum-filtered and concentrated under reduced pressure on a rotary evaporator. Isopropyl alcohol was added during the concentration step to increase the rate of water removal by taking advantage of the azeotrope that forms with water. The extracted material was dried overnight in a Savant-Speed Vac concentration system at 55° C. resulting in weight yields varying between 0.2% and 22.9%. The yield data is shown in Table 1.

TABLE 1
Yield, DPPH, Ferrozine and Polarity Test Results.

| | | | % DPPH | % Ferrozine | |
|---|---|---|---|---|---|
| | | | (100 ppm) ± | (1000 ppm) ± | Polarity ± |
| Extract | Spice | Yield | std. dev. | std. dev. | std. dev. |
| 01 | Clove (wash 1) | 4.6% | (95 ± 1)% | (43 ± 5)% | 2.01 ± 0.8 |
| 02 | Clove (wash 2) | 3.8% | (95 ± 1)% | (42 ± 5)% | 0.8 ± 0.37 |
| 03 | Clove (wash 1) | | | | |
| 04 | Clove (wash 2) | | | | |
| 05 | *Clove (wash 3) | | | | |
| 06 | Clove (wash 4) | | | | |
| 07 | Allspice (wash 1) | 1.7% | (95 ± 1)% | (46 ± 6)% | 0.21 ± 0.02 |
| 08 | Allspice (wash 2) | 4.3% | (94 ± 1)% | (71 ± 2)% | 0.26 ± 0.07 |
| 09 | Carrot (wash 1) | 4.6% | (0-5)% | (32 ± 4)% | 0.06 ± 0.02 |
| 10 | Carrot (wash 2) | 16.1% | (0-5)% | (59 ± 4)% | 0.03 ± 0.00 |
| 11 | *Carrot (wash 1) | 22.9% | (0-5)% | (42.6%) | |
| 12 | Rosemary (wash 1) | 1.2% | (86 ± 7)% | (20 ± 5)% | 1.00 ± 0.05 |
| 13 | Rosemary (wash 2) | 4.2% | (94 ± 1)% | (41 ± 6)% | 0.36 ± 0.04 |
| 14 | Oregano (wash 1) | 6.2% | (62 ± 10)% | (42 ± 5)% | 0.28 ± 0.12 |
| 15 | Oregano (wash 2) | 1.0% | (79 ± 10)% | (61 ± 6)% | 0.27 ± 0.07 |
| 16 | Paprika (wash 1) | 3.9% | (6 ± 3)% | (25 ± 5)% | 0.22 ± 0.09 |
| 17 | Paprika (wash 2) | 9.5% | (8 ± 4)% | (40 ± 7)% | 0.14 ± 0.05 |
| 18 | Hops (wash 1) | 7.2% | (36 ± 3)% | (60 ± 5)% | 2.0 ± 0.15 |
| 19 | Hops (wash 2) | 8.1% | (41 ± 7)% | (55 ± 8)% | 0.39 ± 0.08 |
| 20 | White pepper (wash 1) | 0.64% | (18 ± 2)% | (7 ± 7)% | 1.7 |
| 21 | White pepper (wash 2) | 0.41% | (26 ± 5)% | (27 ± 14)% | 1.04 |
| 22 | Chaff (wash 1) | 1.2% | (11 ± 4)% | (19 ± 4)% | 0.84 ± 0.09 |
| 23 | Chaff (wash 2) | 2.4% | (11 ± 1)% | (22 ± 3)% | 0.24 ± 0.09 |
| 24 | Celery seed (wash 1) | 2.9% | (40 ± 5)% | (61 ± 10)% | 0.12 ± 0.6 |
| 25 | Celery seed (wash 2) | 5.6% | (36 ± 6)% | (54 ± 1)% | 0.27 ± 0.14 |
| 26 | Coriander seed (wash 1) | 1.7% | (19 ± 2)% | (58 ± 4)% | 0.26 ± 0.06 |
| 27 | Coriander seed (wash 2) | 3.9% | (13 ± 3)% | (61 ± 10)% | 0.11 ± 0.04 |
| 28 | Anise seed (wash 1) | 4.3% | (19 ± 3)% | (65 ± 13)% | 2.59 |
| 29 | Anise seed (wash 2) | 7.2% | (19 ± 4)% | (62 ± 11)% | 0.13 ± 0.01 |
| 30 | Cassia (wash 1) | 8.3% | (93 ± 1)% | (2 ± 1)% | 0.69 ± 0.08 |
| 31 | Cassia (wash 2) | 8.5% | (94 ± 1)% | (19 ± 11)% | 0.32 ± 0.16 |
| 32 | Nutmeg (wash 1) | 2.3% | (63 ± 10)% | (52 ± 14)% | 0.49 ± 0.08 |
| 33 | Nutmeg (wash 2) | 4.9% | (79 ± 0)% | (61 ± 4)% | 0.17 ± 0.05 |
| 34 | Cardamom (wash 1) | 0.2% | (13 ± 0)% | (88 ± 1)% | 0.50 |
| 35 | Cardamom (wash 2) | 1.6% | (9 ± 2)% | (89 ± 6)% | 0.13 |
| 36 | Dill seed (wash 1) | 1.8% | (38 ± 3)% | (57 ± 4)% | 0.35 |
| 37 | Dill seed (wash 2) | 4.5% | (30 ± 3)% | (66 ± 1)% | 0.1 |
| 38 | Marjoram (wash 1) | 2.3% | (81 ± 5)% | (31 ± 1)% | 0.68 |
| 39 | Marjoram (wash 2) | 8.7% | (95 ± 2)% | (61 ± 4)% | 0.24 |
| 40 | EDTA | | | | |
| 41 | Purified rosmarinic acid | | | | |
| 42 | Purified carnosic acid | | | | |
| 43 | HERBALOX QS, NS | | | | |
| 44 | HERBALOX 41-19-32 | | | | |

### Example 2

### A Multiple Extraction Method of Preparation

Spent clove, ground and dried, which had originally been extracted commercially with a mixture of hexane and acetone (65 pounds) was placed in the basket of an extractor, designed to allow for solvent to be sprayed onto the basket and for the extract liquid to drain through the bed of plant material and be collected below the basket. A total of 195 pounds of methanol (food grade) was sprayed onto the spent clove biomass and allowed to drain through the plant material bed. A total of 155 pounds of miscella (extract solution) was collected and removed from the extractor. A second extraction was then conducted in a similar manner, with a solvent mixture consisting of 292.5 pounds of methanol and 32.5 pounds of water. The weight of miscella collected from this extraction was 326 pounds. A third extraction was then done in a similar manner, with a solvent mixture consisting of 292.5 pounds of methanol and 32.5 pounds of water. The weight of miscella collected from this extraction was 329 pounds. A fourth extraction was then done in a similar manner, with a solvent mixture consisting of 292.5 pounds of methanol and 32.5 pounds of water. The weight of miscella collected from this extraction was 326.5 pounds. The solvent was removed from these individual extracts, separately, giving the extracts 03, 04, 05 and 06, with the yield data shown in Table 1. Each extract exhibited different color and different solubility characteristics. Certain fractions had surprising dispersibility in lipidic or oil-based systems. The fractions may be re-combined in proportion to their yield weights to provide a total spent clove extract.

### Example 3

### Metal Chelation Screening Assay—Ferrozine Assay

Stock solutions of antioxidant extracts at_{—}10,000 ppm concentration were made by dissolving 0.1 g, separately, of each extract in 1.0 mL of deionized water and sonicating for 10 minutes. Stock solutions of antioxidant extracts at 1,000 ppm concentration were made by combining 4.5 mL of MeOH with 0.5 mL of each of the 10,000 ppm stock solutions. A 5 mM ferrozine stock solution was prepared by dissolving 0.0614 g of ferrozine in 25 mL of deionized H₂O. A 2 mM ferrous sulfate stock solution was prepared by dissolving 0.1112 g of ferrous sulfate in 200 mL deionized H₂O. 5.0 mL of each of the 1000 ppm extract solutions was combined with 167 μL of the ferrous sulfate solution and shaken for 10 seconds. Ferrozine stock solution was then added (335 μL) and the solution was shaken for 5 seconds and incubated at room temperature for 10 minutes. The spectral background of the spectrophotometer was zeroed using HPLC grade MeOH, and the absorbance of the extract solutions with added ferrous sulfate was measured at 562 nm. The absorbance of the control (5.0 mL MeOH added to 167 μL of the ferrous sulfate solution and 335 μL of the ferrozine solution) was measured as well at 562 nm. The absorbance of the extract solutions in MeOH (at 1,000 ppm) was measured at 562 nm before the addition of ferrous sulfate and ferrozine, and subtracted from the value of the absorbance resulting from the addition of ferrous sulfate and ferrozine at 562 nm in order to correct for the absorbance occurring from the actual color of the extract at the tested concentration which might interfere with the absorbance at the same wavelength due to the ferrozine-Fe(II) complex. A percent ferrozine inhibition was determined for each of the extracts at the concentration at which it was tested as follows: % Ferrozine inhibition=[1−(A_{extract}/A_{blank})]×100. A_{extract} being the absorbance at 562 nm of the extract after adding the assay reagents and incubation and A₀ being the absorbance at 562 nm of the control. The Ferrozine Assay results are shown in column 5 of Table 1.

### Example 4

### DPPH Radical Scavenging Screening Assay

A DPPH stock solution was prepared by dissolving 38-40 mg of DPPH in 100 mL of MeOH to yield a 1 mM solution. The DPPH solution was sonicated to insure complete dissolution and was prepared fresh the day it was used. Stock solutions of the extracts at 10,000 ppm concentration were prepared by dissolving 0.1 g of each extract in 1.0 mL of deionized water. The resulting mixtures were sonicated to insure complete dissolution. Stock solutions of the extracts at 10,000 ppm concentration were prepared by adding 100 μL of each of the 10,000 ppm stock solutions to 9.0 mL of MeOH. Extracts that exhibited more than 79% DPPH scavenging at 100 pm were further diluted to 10 ppm by combining 1.0 mL of the 100 ppm solutions with 9.0 mL of MeOH. 10 mL of each of the 100 ppm extract solutions was combined with 1.0 mL of the DPPH solution and incubated at room temperature for 10 minutes. The spectral background of the spectrophotometer was zeroed using HPLC grade MeOH, and the absorbance of the extract solutions with added DPPH was measured at 515 nm. The absorbance of the control (1.0 mL of the DPPH solution added to 10 mL MeOH) was measured at 515 nm as well. A percent DPPH inhibition was determined for each of the extracts at the concentration at which it was tested as follows: % DPPH inhibition=[1−(A_{extract}/A_{blank})]×100. A_{extract} being the absorbance at 515 nm of the extract after reaction with DPPH and incubation for 10 minutes at room temperature and A₀ being the absorbance at 515 nm of the control. The DPPH assay results are shown in column 4 of Table 1.

### Example 5

### OSI Test for Screening Efficacy in Bulk Oil Systems

A 500 μM stock solution of iron (II) chloride in canola oil was prepared by dissolving, with the aid of an ultrasonic bath, 7 mg of iron(II) chloride in 1.0 mL of absolute EtOH, followed by dilution with 100 g of unfortified canola oil. Ethanolic extract stock solutions at 4% concentration were prepared by dissolving or suspending 0.16 g of each of extract to be tested in 4.0 mL of ethanol. The cloudy suspensions were sonicated to insure maximum dissolution. The extract stock solutions (2.0 mL) were added separately to 40 g of canola and shaken for 5 seconds to yield 2,000 ppm solutions of extracts in oil. The oil/extract stock solutions (2.0 mL) were separately added to 32 g of canola and 8.0 g of the Fe(II) stock solution and then shaken for 5 seconds to yield 2,000 ppm extract containing iron. A control oil solution (absent iron) was prepared by adding 2.0 mL of absolute EtOH to 40 g of canola oil. A control oil solution (with iron) was prepared by adding 2.0 mL of absolute EtOH to 32 g of canola oil and 8.0 g of the Fe(II) stock solution. Assays were performed on an OMNION, Inc. OSI instrument according to the AOCS Method Cd 1 2b-92 where duplicate 5-gram samples of the oils to be tested were placed in glass tubes held at a constant temperature (90° C.). Air was bubbled at a flow rate of 150 cc/minute (under constant pressure of 6.5 PSI) through each of the samples, and subsequently was bubbled through a reservoir of deionized water. Conductivity was measured by electrodes in each reservoir, and the induction time determined graphically by the instrument. Incubation was initiated for 30 minutes before connecting the tubes to the water reservoir in order to evaporate the EtOH to avoid any interference with conductivity. Multiple samples were tested simultaneously (up to 24 sample per experiment). OSI results are shown in columns 4 and 5 of Table 2.

TABLE 2
Conjugated diene hydroperoxide (CF_{50 ratio}), carotenoid
color fading (SF) and OSI test results.

| | | | | OSI | OSI |
|---|---|---|---|---|---|
| | CF₅₀ ± Std. Dev. | | SF ± Std. Dev. | (w/o Fe) | (w/Fe) |
| Extract | (ppm extract) | | (ppm extract) | (2000 ppm) | (2000 ppm) |
| Control | | | | 1.0 | 0.16 |
| 01 | | | | 1.34 | 1.21 |
| 02 | | | | | |
| 03 | 5.4 ± 0.4 | (15 ppm) | 0.25 ± 0.02 (500 ppm) | | |
| 04 | 6.3 ± 0.4 | (15 ppm) | 0.23 ± 0.02 (500 ppm) | | |
| 05 | 6.6 ± 1.3 | (15 ppm) | 0.22 ± 0.02 (500 ppm) | | |
| 06 | 5.1 ± 0.5 | (15 ppm) | 0.20 ± 0.02 (500 ppm) | | |
| 07 | 16.6 ± 1.4 | (45 ppm) | 0.29 ± 0.05 (500 ppm) | 1.33 | 1.17 |
| 08 | | | 0.27 ± 0.05 (500 ppm) | | |
| 09 | 1.6 ± 0 | (45 ppm) | | | |
| 10 | 2.2 ± 0.2 | (45 ppm) | | | |
| 11 | | | | | |
| 12 | 4.4 ± 0.8 | (45 ppm) | 0.52 ± 0.01 (500 ppm) | | |
| 13 | 7.1 ± 0.4 | (45 ppm) | 0.33 ± 0.01 (500 ppm) | | |
| 14 | 4.3 + 0.3 | (45 ppm) | 0.54 ± 0.04 (500 ppm) | 1.19 | 1.19 |
| 15 | 4.9 ± 0.8 | (45 ppm) | 0.37 ± 0.06 (500 ppm) | | |
| 16 | 2.5 ± 0.1 | (45 ppm) | 0.67 ± 0.01 (500 ppm) | 1.09 | 1.06 |
| 17 | 4.5 ± 0.1 | (45 ppm) | 0.45 ± 0.02 (500 ppm) | | |
| 18 | 3.4 ± 0.2 | (45 ppm) | 0.65 + 0.03 (500 ppm) | | |
| 19 | 5.7 ± 0.1 | (45 ppm) | 0.41 ± 0.01 (500 ppm) | | |
| 20 | 2.6 ± 0.2 | (45 ppm) | 0.86 ± 0.03 (500 ppm) | 0.87 | 0.53 |
| 21 | 2.6 ± 0.1 | (45 ppm) | 0.76 ± 0.04 (500 ppm) | | |
| 22 | 2.0 ± 0.1 | (45 ppm) | | 1.06 | 0.84 |
| 23 | 3.2 ± 0.0 | (45 ppm) | 0.48 ± 0.04 (500 ppm) | | |
| 24 | 4.0 ± 0.1 | (45 ppm) | 0.72 ± 0.05 (500 ppm) | | |
| 25 | 4.1 ± 0.2 | (45 ppm) | 0.49 ± 0.05 (500 ppm) | | |
| 26 | 1.4 | (45 ppm) | 0.84 ± 0.02 (500 ppm) | 1.09 | 0.76 |
| 27 | 1.4 | (45 ppm) | 0.67 ± 0.01 (500 ppm) | | |
| 28 | 2.8 | (45 ppm) | 0.83 ± 0.02 (500 ppm) | 1.06 | 0.97 |
| 29 | 2.8 | (45 ppm) | 0.54 ± 0.03 (500 ppm) | | |
| 30 | 15.5 | (45 ppm) | 0.33 ± 0.02 (500 ppm) | 1.09 | 0.74 |
| 31 | 14.1 | (45 ppm) | 0.34 ± 0.02 (500 ppm) | | |
| 32 | 5.6 | (45 ppm) | 0.73 ± 0.04 (500 ppm) | 1.11 | 0.74 |
| 33 | 7.1 | (45 ppm) | 0.39 ± 0.02 (500 ppm) | | |
| 34 | 1.3 | (45 ppm) | 0.92 | | |
| 35 | 1.1 | (45 ppm) | 0.91 | | |
| 36 | 1.4 | (45 ppm) | 0.92 | | |
| 37 | 1.2 | (45 ppm) | 0.85 | | |
| 38 | 1.9 | (45 ppm) | 0.84 | | |
| 39 | 2.8 | (45 ppm) | 0.41 | | |
| 40 | 13.4 ± 0.06 | (1 ppm) | 0.45 ± 0.1 (100 ppm) | | |
| 41 | 5.4 | (10 ppm) | | | |
| 42 | 2.7 | (10 ppm) | | | |
| 43 | 1.7 | (15 ppm) | | | |
| 44 | 1.5 | (15 ppm) | 0.6-0.7 (500 ppm) | | |

### Example 6

### Carotenoid Color Fading Test for Screening Efficacy of Antioxidant Systems

A 50 ppm solution of paprika oleoresin in H₂O containing 1% EtOH was prepared by dissolving 50 mg of paprika oleoresin in 10 mL of absolute EtOH. The EtOH solution is subsequently added to 990 mL of deionized H₂O to yield 1 L of 50 ppm paprika oleoresin solution in H₂O (1% EtOH). To prepare a stock solution of 10,000 ppm concentration, 0.01 g of the extract was ultrasonically dissolved in 1 mL of deionized H₂O. A 1,000 ppm stock solution of EDTA was prepared by dissolving 0.001 g of EDTA in 1 mL of deionized H₂O. Stock solutions of 10,000 ppm concentration of two types of commercially available rosemary extracts (HERBALOX® Seasoning QS, (41-19-49) and HERBALOX® Seasoning (41-19-49) were prepared by dissolving 0.01 g of each in 1 mL of deionized H₂O. Using a micropipette, 90 μL, each, of the 10,000 ppm stock solutions of the antioxidant extracts and HERBALOX® (41-19-49) was added separately to 20 mL of the 50 ppm paprika solution in H₂O (1% EtOH) and vortexed for 5 seconds, giving extract/paprika oleoresin solutions containing 45 ppm of antioxidant extracts. These solutions were made up in triplicate. Similarly, extract/paprika oleoresin solutions containing 15 ppm of the antioxidant extracts were prepared in triplicate using 30 μL, of the 10,000 ppm stock solutions. To make a 10 ppm antioxidant in paprika oleoresin color solution, 20 μL of the 10,000 ppm stock solution of each of: rosmarinic acid and carnosic acid was added to 20 mL of the 50 ppm paprika solution in H₂O (1% EtOH) and vortexed for 5 seconds. Again, all the 10 ppm antioxidant in paprika oleoresin color solutions were made up in triplicate. Solutions of EDTA a 1 ppm concentration in paprika oleoresin color solution were prepared by adding 20 μL of the 1,000 ppm stock solution of EDTA to 20 mL of the 50 ppm paprika solution in H₂O (1% EtOH) and vortexing for 5 seconds. The 1 ppm EDTA in paprika oleoresin color solution was prepared in triplicate. Control solutions were prepared by adding 20, 30 and 90 μL of deionized H₂O to 20 mL of the 50 ppm paprika solution in H₂O (1% EtOH) and vortex mixing for 5 seconds. All the paprika color solutions treated with extracts/antioxidants were incubated in scintillation vials placed in a “GALLENKAMP Plus Oven” oven set at 45° C., and were taken out for 10 minutes every day. After cooling to room temperature, absorbance measurements were taken, and then the scintillation vials containing the treatment solutions were returned to the oven. Experiments were performed on all replicates. The initial absorbance was measured on a Beckman-Coulter DU® 800 UV/Vis Spectrophotometer using a 1 cm cell. A visible wavelength scan was used to determine the wavelength (λₘₐₓ) at which the maximum absorbance occurred (460 nm). The absorbance of the antioxidant treatments in paprika oleoresin solutions was corrected for the absorbance due to the sample alone at the wavelength of maximum absorbance. Absorbance (at λₘₐₓ=460 nm) measurements were recorded at 24 hr intervals, until the color visually faded in the solution. For each antioxidant/extract treated paprika oleoresin solution, the absorbance (at λₘₐₓ=460 nm) was plotted against time, and the time corresponding to 50% color fading (hereafter referred to as CF₅₀) was determined graphically from the equation of the pseudo-straight line comprising three data points around the CF₅₀. Subsequently, CF₅₀ of each solution treated with an extract/antioxidant was divided by the CF₅₀ of the control, yielding for each antioxidant/extract treated paprika oleoresin color solution; a CF₅₀ ratio (CF₅₀ ratio=1 for control). The results are listed in column 2 of Table 2.

### Example 7

### Oil in Water (O/W) Emulsion Screening Test

100 g of unfortified canola oil was mixed with 400 mL of deionized water and 10 g of polysorbate 20 (Tween 20) using a WARING® Blender. The blended emulsion was passed through a PVA single-stage homogenizer 10 times, and then stored refrigerated at 6° C. 10,000 ppm stock solutions of antioxidant extracts were made by dissolving 0.1 g of the antioxidant extracts in 1.0 mL of deionized water and sonicating for 10 minutes. A 1,000 ppm stock solution of EDTA was made by dissolving 0.01 g of EDTA in 1.0 mL of deionized water. Emulsion solutions containing 1,000 ppm of antioxidant extract were prepared by adding 30 μL of the extract stock solution of each antioxidant to 3.0 mL of the O/W emulsion and vortex mixing for 5 seconds, followed by sonication for 1 minute. Similarly, emulsion solutions containing 500, 250 and 100 ppm of the antioxidant extracts were prepared using 15, 7.5 and 3 μL of the extract stock solutions, respectively. All 20% O/W emulsion treatments dosed with extracts were incubated at 60° C. on an orbital shaker along with control treatments consisting of 3.0 mL emulsion with 3, 7.5, 15 and 30 μL of deionized water replacing the extract stock solutions. Measurements were taken once a day, for seven consecutive days by sampling 20 μL of each emulsion treatment and transferring into 10 mL of 2-propanol for analysis. The UV absorbance of the conjugated dienes was measured at 234 nm. Experiments were done in triplicate. For each antioxidant/extract treated emulsion solution, the absorbance (at λ=234 nm) was plotted against time, and the slope was determined graphically from the equation of the pseudo-straight line corresponding to the appearance of conjugated dienes. Subsequently, the slope of each treatment with an extract/antioxidant graph was divided by the slope of the control, yielding for each antioxidant/extract treated emulsion solution a stabilization factor (SF). The results are listed in column 3 of Table 2.

### Example 8

### Water in Oil (W/O) Emulsion Screening Test

A control sample is prepared by mixing 100 mL of deionized water with 400 g of unfortified canola oil, 8.0 g of Atmos 300 K and 1 g of Tween 20 using a WARING® Blender. The blended emulsion is passed through a single stage homogenizer 10 times, and then stored refrigerated at 6° C. Test samples containing the inventive antioxidant metal chelating compositions, optionally containing one or more non-chelating antioxidant component also derived from edible herbs, spices, fruits, vegetables and/or grains, and/or further optionally combined with one or more synthetic food grade antioxidants are prepared by adding the antioxidant to the water or the oil used to make the emulsion, and the W/O emulsions are prepared as described above. The W/O emulsions are allowed to age, aliquots are taken a various times and dissolved in methanol or ethanol with sonication. The absorbance at 234 nm is measured and it is found that conjugate diene hydroperoxide formation occurs much more slowly in the antioxidant-treated emulsions than in the controls.

### Example 9

### Measuring Partition Ratios for Spent Extracts, and Showing Extracts can be Further Partitioned

A test was established to determine the partition coefficient of the plant extracts between two solvents of different polarity. This test served primarily as an indication of the polarity and water solubility of the tested plant extracts. Subsequent assays consisted of testing the separated mass fractions of each extract independently by the DPPH assay and Ferrozine Assay described earlier, to determine the radical scavenging and metal chelating activity. These assays constituted a tool to separate the constituents of the extract, determine the partition between two solvents of different polarities as well as water solubility, and determine the radical scavenging potential and metal chelating potential of the separated phases.

Each antioxidant extract (0.4 g) was dissolved with the aid of sonication in a mixture of 4.0 mL of deionized H₂O (polar) and 4 mL of 1-butanol (BuOH, less polar). 1.0 mL was taken from each phase (water and butanol) and dried overnight in a pre-weighed scintillation vial in a Savant-Speed Vac concentration system at 65° C. The mass of the extract material partitioned in each phase was weighed the next day after drying. A partition coefficient between H₂O/BuOH was determined as: coefficient=mass in BuOH/mass in H₂O. Subsequently, each of the partitioned dried extracts was tested by both the DPHH test and the Ferrozine Assay.

### DPPH Test

A 1 mM solution of DPPH was prepared by dissolving 38-40 mg of DPPH in 100 mL of MeOH, assisted by sonication. The DPPH solution was prepared fresh the day it was used. Stock solutions of the partitioned, dried extracts at 10,000 ppm were prepared by separately dissolving 0.1 g of the residue isolated from each phase from each initial antioxidant extract in 1.0 mL of deionized water. Stock solutions of the partitioned dried extracts at 100 ppm concentration were prepared by adding 100 μL of each of the 10,000 ppm stock solutions to 9.0 mL of MeOH. Partitioned, dried extracts that exhibited more than 79% DPPH scavenging at 100 pm were further diluted to 10 ppm by adding 1.0 mL of the 100 ppm solutions to 9.0 mL of MeOH. To perform the test, 10 mL of each of the 100 ppm partitioned, dried extract solutions was combined with 1.0 mL of the DPPH solution and incubated at room temperature for 10 minutes. The spectral background of the spectrophotometer was zeroed using HPLC grade MeOH, and the absorbance of the extract solutions with added DPPH was measured at 515 nm. The absorbance of the control (1.0 mL of the DPPH solution added to 10 mL MeOH) was measured at 515 nm as well. A percent DPPH inhibition was determined for each of the extracts at the concentration at which it was tested as follows: % DPPH inhibition=[1−(A_{extract}/A_{blank})]×100. A_{extract} being the absorbance at 515 nm of the extract after reaction with DPPH and incubation for 10 minutes at room temperature and A₀ being the absorbance at 515 nm of the control.

### Ferrozine Assay

Stock solutions of the partitioned, dried extracts at 1,000 ppm concentration were prepared by 4.5 mL of MeOH with 0.5 mL of each of the 10,000 ppm stock solutions described, above. A 5 mM ferrozine stock solution was prepared by dissolving 0.0614 g of ferrozine in 25 mL deionized H₂O. A 2 mM ferrous sulfate stock solution was prepared by dissolving 0.1112 g of ferrous sulfate in 200 mL deionized H₂O. 5.0 mL of each of the 1000 ppm extract solutions was combined with 167 μL of the ferrous sulfate solution and shaken for 10 seconds. Ferrozine stock solution was then added (335 μL) and the solution was shaken for 5 seconds and incubated at room temperature for 10 minutes. The spectral background of the spectrophotometer was zeroed using HPLC grade MeOH, and the absorbance of the extract solutions with added ferrous sulfate was measured at 562 nm. The absorbance of the control (5.0 mL MeOH added to 167 μL of the ferrous sulfate solution and 335 μL of the ferrozine solution) was measured as well at 562 nm. The absorbance of the extract solutions in MeOH (at 1,000 ppm) was measured at 562 nm before the addition of ferrous sulfate and ferrozine, and subtracted from the value of the absorbance resulting from the addition of ferrous sulfate and ferrozine at 562 nm in order to correct for the absorbance occurring from the actual color of the extract at the tested concentration which might interfere with the absorbance at the same wavelength due to the ferrozine-Fe(II) complex. A percent ferrozine inhibition was determined for each of the extracts at the concentration at which it was tested as follows: % Ferrozine inhibition=[1−(A_{extract}/A_{blank})]×100. A_{extract} being the absorbance at 562 nm of the extract after adding the assay reagents and incubation and A₀ being the absorbance at 562 nm of the control. The results are shown in Table 3.

TABLE 3
Partition Coefficients, DPPH and Ferrozine
Inhibition of Partitioned Materials.

| | | DPPH of | DPPH of | Ferrozine of | Ferrozine of |
|---|---|---|---|---|---|
| Ex- | Polarity ± | H₂O | BuOH | H₂O | BuOH |
| tract | Std. Dev. | (100 ppm) | (100 ppm) | (1000 ppm) | (1000 ppm) |
| 01 | 2.01 ± 0.8 | 38% | 29.3% | 79.3% | 13.6% |
| 02 | 0.8 ± 0.37 | 39.2% | 60.8% | 80.7% | 28.6% |
| 03 | | | | | |
| 04 | | | | | |
| 05 | | | | | |
| 06 | | | | | |
| 07 | 0.21 ± 0.02 | 40.8% | 46.1% | 53.9% | 23.34% |
| 08 | 0.26 ± 0.07 | 36.6% | 50.9% | 73.7% | 6.7% |
| 09 | 0.06 ± 0.02 | 5.1% | 22.5% | 50.2% | 45.3% |
| 10 | 0.03 ± 0.00 | 5.5% | 24.7% | | |
| 11 | | | | | |
| 12 | 1.00 ± 0.05 | 13.9% | 14.5% | | |
| 13 | 0.36 ± 0.04 | 17.8% | 23.4% | | |
| 14 | 0.28 ± 0.12 | 45.3% | 9.8% | 62.2% | 13.1% |
| 15 | 0.27 ± 0.07 | 11.9% | 28.8% | 66.5% | 23.5% |
| 16 | 0.22 ± 0.09 | 8.9% | 24.2% | 70.48% | 24.54% |
| 17 | 0.14 ± 0.05 | 9.6% | 28.5% | 84.8% | 45.3% |
| 18 | 2.0 ± 0.15 | 23.3% | 25.01% | 88.1% | 86.7% |
| 19 | 0.39 ± 0.08 | 29.8% | 43.0% | 92.2% | 70.9% |
| 20 | 1.7 | 8.07% | 19.63% | 70.2% | 6.7% |
| 21 | 1.04 | 9.67% | 33.15% | 67.7% | 1.6% |
| 22 | 0.84 ± 0.09 | 7.1% | 29.8% | 38.3% | 20.4% |
| 23 | 0.24 ± 0.09 | 7.9% | 36.9% | 40.9% | 17.7% |
| 24 | 0.12 ± 0.6 | 30.9% | 47.2% | 53.2% | 0.0% |
| 25 | 0.27 ± 0.14 | 24.6% | 76.7% | 44.5% | 48.0% |
| 26 | 0.26 ± 0.06 | 30.0% | 47.6% | 67.1% | 0.0% |
| 27 | 0.11 ± 0.04 | 13.4% | 53.5% | 60.0% | 9.7% |
| 28 | | 12.2% | 90.0% | 59.6% | 42.6% |
| 29 | 0.13 ± 0.01 | 16.3% | 79.5% | 62.4% | 30.3% |
| 30 | 0.69 ± 0.08 | 95.0% | 93.9% | 4.2% | 0.0% |
| 31 | 0.32 ± 0.16 | 94.1% | 94.3% | 24.4% | 0.0% |
| 32 | 0.49 ± 0.08 | 69.9% | 83.1% | 43.5% | 0.1% |
| 33 | 0.17 ± 0.05 | 50.9% | 87.2% | 45.02% | 4.2% |
| 34 | 0.50 | 6.8% | 28.8% | 89.2% | 28.3% |
| 35 | 0.13 | 0% | 50.2% | 88.7% | 34.3% |
| 36 | 0.35 | 31.6% | 62.9% | 70.1% | 14.1% |
| 37 | 0.1 | 0% | 78.8% | 73.2% | 23.1% |
| 38 | 0.68 | 80.1% | 84.3% | 41.6% | 23.2% |
| 39 | 0.24 | 82.2% | 81.5% | 23.2% | 7.8% |

### Example 10

### Showing the Effect of Chelating Composition on the Prevention of Warmed Over Flavor and Enhancement of Microbiological Shelf Life of Cooked Meats

An accelerated (38° F., 11 day) shelf-life test examining lipid oxidation and microbiological stability was conducted on 80% lean precooked, uncured ground beef stored at 38° F. in polyethylene non-barrier bags. One 10 lb. chub of fresh ground 80% lean ground beef was purchased from a local supermarket. The ground beef chub was held at 32° F. for 12 hours prior to processing. Two pounds (908 grams) was used for the control (without clove extract) and two pounds was used for the treatment (with clove extract 05—see Table 1). These were separately placed into a stainless steel, pre-chilled bowl and mixed with a KITCHENAID® mixer for 1½ minutes in a 38° F. processing room. After mixing, the meat was ground on a Biro #812 grinder through a 3/16″ plate and 4-winged knife. Burger patties were portioned using a stainless steel patty former into 130 g portions, cooked at 300° F. to an internal temperature of 160-165° F. The cooked portions were chilled at 38° F. and then placed into non-barrier polyethylene bags and stored at 38° F. for 11 days for microbiological evaluation; and, for 48 hours for evaluation for lipid oxidation. Two portions from each treatment were frozen in a sharp freezer at −25° F., vacuum packaged and stored at −15° F. as samples for time zero. Lipid oxidation (as measured by TBARS) and microbiological assays (aerobic and psychrotrophic plate counts) were conducted. Thiobarbituric Acid Reactive Substance (TBAR) testing was done to measure the extent of oxidation. An identical sample of ground beef was prepared, cooked and stored, except that the additive was HERBALOX® Seasoning Type 25 at 2000 ppm. Absorbance readings for the TBAR test were made in duplicate at 531 nm. These reading were averaged and used to calculate the TBAR value. The results are shown in Table 4.

TABLE 4
TBAR values for cooked ground beef held at 38 degrees F.

| | TBAR Value | TBAR Value |
|---|---|---|
| Treatment | Time 0 | Time 48 hr |
| Control | 1.49 | 3.45 |
| HERBALOX ® Seasoning | 0.28 | 1.25 |
| Type 25 (2000 ppm) | | |
| 100 ppm spent clove | 0.20 | 0.26 |
| extract | | |
| 250 ppm spent clove | 0.15 | 0.16 |
| extract | | |
| 500 ppm spent clove | 0.13 | 0.13 |
| extract | | |

This data shows that spent clove extract is much more efficient at controlling oxidation (maintaining low TBAR values), than the current state of the art product HERBALOX® Seasoning Type 25.

Microbiological testing, consisting of aerobic and psychrotroph counting, was performed on a composite sample of all ground beef treatments at time zero and on the individual samples at day eleven. The data is shown in Table 5.

TABLE 5
Microbiological Data.

| | Aerobic Plate Count | Psychrotrophs |
|---|---|---|
| Treatment | (cfu/g) | (cfu/g) |
| Composite sample, time = 0 | 100,000 | 570,000 |
| Control, time 11 days | 68,000,000 | 75,000,000 |
| 100 ppm spent clove | 27,000,000 | 32,000,000 |
| extract, time = 11 days | | |
| 250 ppm spent clove | 20,000,000 | 26,000,000 |
| extract, time = 11 days | | |
| 500 ppm spent clove | 26,000,000 | 35,000,000 |
| extract, time = 11 days | | |
| HERBALOX ® Type 25, | 14,000,000 | 16,000,000 |
| time = 11 days | | |

This data shows that, surprisingly, the clove chelator is acting as an antimicrobial agent and that the lowest dose tried is as effective as the highest dose. It is possible that a rosemary/clove blend may be even more effective.

### Example 11

### Showing the Stabilizing Effect of Chelating Compositions on Cured Meat Oxidation (Flavor) and Color

Clove extract was prepared from dried clove material that had been previously extracted with non-polar solvents to remove flavor and essential oils using a 9:1 ratio of methanol and water in a solvent to spice ratio of 3:1. The sample used was the Extract 05 from Table 1. Fresh ground beef (75% lean) was purchased from a local supermarket in five, 10 lb. chubs. The ground beef was stored at 32° F.−35° F. in a reach in cooler for 12 hours before processing. Fifty pounds of the ground beef was ground on a Biro #812 grinder through a ⅛″ plate and 4-winged knife. Twenty-five pounds of re-ground beef were placed in a clean, pre-chilled stainless steel MAINC RM#35 paddle/ribbon mixer, 0.20% by weight of maltodextrin M100 (GPC) was added to the ground beef (control), and the ground beef was mixed for 3 minutes. The mixed meat was placed into a plastic tote, placed in 32-35° F. KELVINATOR® reach-in cooler. A second 25 lb. batch of re-ground beef was placed into the MAINCA mixer, and 0.04 pounds of maltodextrin upon which 2.86 g of clove extract had been dispersed was added and the composition was mixed for 3 minutes. All processing (grinding, mixing, stuffing) was conducted in a 38° F. processing room. Four, six pound lots of ground beef from the control and treatment groups were weighed into 10.5″×11″ ZIPLOCK® freezer storage bags and labeled. One bag from treatment and control group were processed into bologna. Three bags from each treatment were placed on coated wire racks in a 10° F. TYLER® walk-in freezer for 0, 1, 3 and 6 months before processing into bologna. The bologna was processed in a programmable, atmosphere controlled RATIONAL® Oven to an internal temperature of 155° F., chilled to below 40° F. overnight then sliced to 3/16″ on a WARING® Pro food slicer and sliced in a 38° F. processing room. Five to seven slices from each treatment were placed in a polyethylene non-barrier ZIPLOCK® bag and 7″×11″ barrier (3 mil, Nylon/PE) pouch (PRIME SOURCE®) or a gas flushed (70% nitrogen and 30% carbon dioxide) sealed pouch. MAPackages were tested for gas ratios and residual oxygen using a Dansensor Checkmate 9900 gas analyzer. Packages of sliced bologna were then placed into a 35° F. reach-in TRUE® cooler and stored for evaluation. The colorimetry data (a*/b* ratios) measuring cure color for never frozen sliced beef sausages is shown in Table 6.

TABLE 6
Colorimetry data for sliced beef sausages
processed from meat never frozen.

| Sample | Mean a */b * | Std. Dev. |
|---|---|---|
| Control Time = 0 * | 1.97 | 0.06 |
| Control Time = 4 weeks * | 1.70 | 0.05 |
| Control Time = 8 weeks * | 1.26 | 0.07 |
| Control Time = 12 weeks * | 1.02 | 0.05 |
| Clove Treatment Time = | 1.99 | 0.08 |
| 0 * | | |
| Clove Treatment Time = | 1.90 | 0.08 |
| 4 weeks * | | |
| Clove Treatment Time = | 1.80 | 0.07 |
| 8 weeks * | | |
| Clove Treatment Time = | 1.59 | 0.07 |
| 12 weeks * | | |
| Control Time = 0 ** | 1.97 | 0.056 |
| Control Time = 4 weeks ** | 2.00 | 0.06 |
| Control Time = 8 weeks ** | 1.92 | 0.06 |
| Control Time = 12 weeks ** | 1.17 | 0.06 |
| Control Time = 18 weeks ** | 0.91 | 0.05 |
| Clove Treatment Time = | 1.99 | 0.08 |
| 0 ** | | |
| Clove Treatment Time = | 1.94 | 0.08 |
| 4 weeks ** | | |
| Clove Treatment Time = | 1.95 | 0.07 |
| 8 weeks ** | | |
| Clove Treatment Time = | 1.80 | 0.07 |
| 12 weeks ** | | |
| Clove Treatment Time = | 1.45 | 0.05 |
| 18 weeks ** | | |

* Stored in gas permeable bags
** Stored in MAP packages

After one month, beef formulation that had been stored in the freezer was thawed and converted into beef sausage as above. Again, the a*/b* ratios were determined to measure color stability. This data is shown in Table 7.

TABLE 7
Colorimetry data for sliced beef sausages processed
from meat stored frozen for one month.

| Sample | Mean a */b * | Std. Dev. |
|---|---|---|
| Control Time = 0 * | 1.94 | 0.07 |
| Clove Treatment Time = 0 * | 2.01 | 0.06 |
| Control Time = 6 weeks ** | 1.93 | 0.05 |
| Control Time = 8 weeks ** | 1.86 | 0.04 |
| Control Time = 13 weeks ** | 1.17 | 0.05 |
| Clove Treatment Time = | 1.97 | 0.07 |
| 6 weeks ** | | |
| Clove Treatment Time = | 1.96 | 0.06 |
| 8 weeks ** | | |
| Clove Treatment Time = | 1.93 | 0.06 |
| 13 weeks ** | | |

* Stored in gas permeable bags
** Stored in MAP packages

After three months, beef formulation that had been stored in the freezer was thawed and converted into beef sausage as above. Again, the a*/b* ratios were determined to measure color stability. This data is shown in Table 8.

TABLE 8
Colorimetry data for sliced beef sausages processed
from meat stored frozen for three months.

| Sample | Mean a */b * | Std. Dev. |
|---|---|---|
| Control Time = 0 * | 1.94 | 0.07 |
| Clove Treatment Time = 0 * | 2.01 | 0.06 |
| Control Time = 4 weeks * | 1.72 | 0.06 |
| Control Time = 8 weeks * | 1.22 | 0.08 |
| Clove Treatment Time = | 1.94 | 0.06 |
| 4 weeks * | | |
| Clove Treatment Time = | 1.78 | 0.05 |
| 8 weeks * | | |
| Control Time = 4 weeks ** | 2.01 | 0.04 |
| Control Time = 8 weeks ** | 1.50 | 0.05 |
| Clove Treatment Time = | 2.01 | 0.06 |
| 4 weeks ** | | |
| Clove Treatment Time = | 1.98 | 0.05 |
| 8 weeks ** | | |

* Stored in gas permeable bags
** Stored in MAP packages

Visual examination of the bag treatments showed that the clove extract, surprisingly, was inhibiting mold on storage.

### Example 12

### Showing a Rapid Screening Test to Examine the Propensity of the Extracts for Form Unwanted Colors in the Presence of Metal Ions

Stock solutions of various spent extracts were prepared at a concentration of 10,000 ppm by diluting 100 mg of extract into 10 mL of distilled water in a scintillation vial. The spent extracts evaluated were clove, allspice, cassia, rosemary, oregano, celery, nutmeg, anise, paprika, mistletoe, coriander and carrot. A stock solution of FeSO₄-7H₂O was made by diluting 199.13 mg into 100 mL of distilled H₂O in a 4 oz glass jar, giving a final concentration of Fe²⁺ of 400 ppm. All of the samples were placed in a sonicator and mixed until dissolved or well-suspended. Test solutions were made of each extract by filling twelve scintillation vials with 18.8 mL of distilled H₂O. To these were added 1.0 mL of each extract and 0.2 mL of FeSO₄-7H₂O stock solution. This yielded final concentrations of 50 ppm of extract and 4 ppm of Fe²⁺. An additional twelve scintillation vials were filled with 19.0 mL of distilled H₂O. To these 1.0 mL of each extract was added to make up a 50 ppm control solution. Each vial was vortex mixed for about 10 seconds and left on the lab bench. The samples were allowed to sit for 30 minutes and inspected for any change in color in the iron treated samples relative to the non-iron treated samples. The iron treated samples were then ordered by color from darkest to lightest. The rank order was (darkest to lightest): clove>allspice>cassia>rosemary>celery>oregano>nutmeg>paprika>anise>mistletoe>coriander>carrot.

### Example 13

### Stabilizing Mayonnaise

The mayonnaise recipe consisted of 80% oil, 8% egg yolk, 6.6% vinegar (at 5% acidity), 3.9% water, 1% sugar and 0.5% salt. A control mayonnaise was prepared by first combining the egg yolk, sugar, salt and water in the mixing bowl for a stand mixer. This was hand-whisked until well mixed. The bowl was then placed on the stand mixer and the mixer set to the second speed. Oil was slowly added while mixing. Vinegar was then added and the mayonnaise was mixed for an additional 2 minutes. Finally, the entire mixture was transferred to a food processor and blended until the desired consistency was achieved. The following test mayonnaise samples were prepared. All test materials were dissolved in either the oil or the vinegar, according to their solubility, for incorporation into the mayonnaise, using the process described for the control.

| Control | No additive |
|---|---|
| Test 1 | 0.05% Carrot Extract 11 (See Table 1) |
| Test 2 | 0.075% Carrot Extract 11 (See Table 1) |
| Test 3 | 0.05% Carrot Extract 11 + 0.025% Herbalox ® Seasoning |
| | 41.088319 |
| Test 4 | 0.075% Carrot Extract 11 + 0.025% Herbalox ® Seasoning |
| | 41.088319 |

Both control and treated samples were packaged into individual units of sufficient size for subsequent testing. Mayonnaise samples were separated into two storage regimes and stored at two different temperatures, 20° C. and 30° C., in the dark for the duration of the study. Samples were analyzed every two weeks for the first 3 months of the study and every 4 weeks for the remaining 6 months. Analysis consisted of headspace gas chromatography/mass spectrometry and sensory analysis by a trained panel. The keeping quality of the mayonnaise was found to be in the following order of increasing stability: Control<Test 1<Test 2<<Test 3˜Test 4. When mayonnaise was prepared using clove extract or allspice extract, the finished product had an unappealing gray color as a result of formation of a dark, finely divided precipitate. HERBALOX® Seasoning 41.088319 is a commercially available, deflavorized, oil soluble rosemary extract containing carnosic acid and carnosol. It is expected that the combination of chelator antioxidants with water soluble rosemary extracts containing rosmarinic acid will also be highly antioxidative and useful in this and other applications. It is further expected that combinations of oil and water dispersible chelator antioxidants (such as a mixture of carrot and anise extract) together with oil and water soluble radical scavengers (such as a mixture of oil and water soluble rosemary extracts) will be highly antioxidative and useful in this and other applications.

### Example 14

### Stable Lipstick Formulation

A lipstick is prepared by combining 6 g beeswax, 3 g carnauba wax, 7 g candelilla wax, 4 g ozokerite, 30 g polyisobutylene. 3 g cosmetic grade mica, 2.25 g red 7 lake and 44.75 g castor oil. The mixture is heated to 80° C. with stirring. 0.3 g of peppermint oil flavoring, 0.03 g of BHT and 0.05 g of tocopherol is added. The mixture is stirred and poured into molds and allowed to cool. A second lipstick is made using the same recipe, but in place of BHT, 0.10 g of the total spent clove extract of Example 2 is added and in place of tocopherol, 0.1 g of HERBALOX® Seasoning 41.088319 is added. The lipstick is allowed to age and the quality of the lipstick is monitored over time using chemical analysis, sensory analysis and microbiological analysis. The lipstick containing clove extract is found to have a longer shelf life and continuously lower microbial count than the lipstick containing BHT.

### Example 15

### Stabilizing an Eye Health Nutritional Supplement Formulation

Beadlets containing 5% zeaxanthin are prepared commercially using a modified food starch. Incorporation of clove extract 05 (Table 1) enhances zeaxanthin storage stability.

### Example 16

### Stabilizing Frying Oil and Fried Foods

Four frying oils are prepared using canola oil. One is treated with 2000 ppm of HERBALOX® Seasoning Type HT-O. Another is treated with 1000 ppm of the total spent clove extract of Example 2. Another is treated with 2000 ppm of HERBALOX® Seasoning Type HT-O and 1000 ppm of the total spent clove extract of Example 2. Another is the untreated control. Multiple batches of previously par-fried sliced potatoes are fried, separately in each of the oils. The quality of the frying oil and the quality of the fried potatoes are monitored as a function of time using chemical analysis and sensory analysis. The treated oils are found to last longer (remain of higher quality) than the untreated oil. The fried potatoes are found to be of higher quality and have a longer shelf life when fried in the treated oils versus the control oil. The highest performing oil and fried food resulted from the treatment package consisting of a combination of 2000 ppm of HERBALOX® Seasoning Type HT-O and 1000 ppm of the total spent clove extract of Example 2. It is a matter of simple experiment to optimize the oil performance using the extracts from Example 1, in combination with added natural and/or synthetic antioxidants.

### Example 17

### Stabilizing Coffee Extracts

A fresh, commercial coffee extract is divided into four portions. One portion serves as the control. A second portion is treated with a mixture of spent clove and spent carrot extracts. A third portion is treated with HERBALOX® 41-19-32, a water soluble rosemary extract containing rosmarinic acid. A fourth portion is treated with a mixture of spent clove and spent carrot extracts and with HERBALOX® 41-19-32, a water soluble rosemary extract containing rosmarinic acid. The coffee extracts are allowed to age and are analyzed using chemical analysis and sensory analysis. The performance, in terms of flavor stability, follows the increasing order: control<HERBALOX® 41-19-32<spent clove and spent carrot extracts<spent clove and spent carrot extracts plus HERBALOX® 41-19-32.

### Example 18

### Stabilizing Beer

Beer is brewed, optionally adding 2000 ppm spent carrot extract 11 (Table 1) to the mash. The finished treated beer has higher flavor stability than the same beer absent the carrot extract.

### Example 19

### Stabilizing an Anthocyanin-Containing Beverage

A strawberry-flavored beverage colored with an anthocyanin-based food color is found to be more stable when it also contains 1000 ppm of spent paprika, spent clove, spent allspice, spent carrot, spent black pepper, spent anise, spent rosemary or spent oregano extract.

### Example 20

### Pilot Scale Extraction of Spent Carrot

Spent carrot from dried, ground carrot tissue that had been previously extracted using a mixture of hexane and acetone (8 lbs.) was extracted a small pilot-scale extraction column using four washes of 100% MeOH at a ratio 1:4.5, spice to solvent. These four washes were combined and gravity filtered through a 1 micron polyester felt filter bag prior to desolventization on the pilot plant Buchi R-220 rotavap. An extract with glassy consistency (952.8 g) was recovered. This was re-dissolved in a 50/50 ethanol/water solution at a ratio of 1:7, extract/solvent, and re-desolventized. The resulting mass was scraped from the round bottom and ground into a coarse powder. The recovery was 831.9 gm with some weight loss due to transfers. The total weight yield of 831.9 gm amounted to a 22.9% extraction yield with a ferrozine activity of 42.62% and corresponds to extract 11 in Table 1.

### Example 21

### Stabilization of Potted Meat Baby Food

A meat emulsion product suitable for use as a baby food is retorted in glass jars. No provision is made to eliminate oxygen from the headspace of the containers. Meat product without added spent clove extract shows a discoloration of the surface of the meat after a storage time. Meat product prepared with 100 ppm of spent clove extract 11 (Table 1) remains free from the discoloration at an identical storage time and under similar storage conditions.

### Example 22

### Stabilizing Emulsions

A freshly prepared 20% oil-in-water emulsion as in Example 7 was divided into four portions. One portion serves as the control (no additives). A second portion was treated with a mixture of spent clove extract (1,000 ppm). A third portion was treated with HERBALOX® 41-19-32 (1,000 ppm), a water soluble rosemary containing rosmarinic acid. A fourth portion is treated with a mixture of spent clove (500 ppm) and HERBALOX® 41-19-32 (500 ppm), a water soluble rosemary extract containing rosmarinic acid. At the initial time of incubation, all four treatments showed a 234 nm absorbance of 0.16. After 120 h of incubation at 60° C., the 234 nm absorbance was 0.81 for the control, 0.27 for the spent clove (1,000 ppm) treated portion, 0.36 for the HERBALOX® 41-19-32 (1,000 ppm) treated portion and 0.26 for the spent clove (500 ppm) and HERBALOX® 41-19-32 (500 ppm) mixture. The higher the absorbance, the higher the concentration of the conjugated hydroperoxides (a sign of a higher level of oxidation). An additive effect of the two extracts in the same portion (spent clove with HERBALOX® 41-19-32) should have manifested in an absorbance close to half the value of the individual absorbance of each (0.36+0.27)/2=0.32. Surprisingly, the absorbance of the portion treated with a mixture of spent clove and HERBALOX® 41-19-32 was 0.26, which can be attributed to a synergistic effect of the two extracts.

1. A plant extract composition derived from previously extracted edible herbs, spices, fruits and/or vegetable matter which has been depleted of the majority of oil-soluble constituents, wherein the plant extract composition exhibits metal chelating activity.

2. The plant extract composition of claim 1, wherein the previously extracted edible herbs, spices, fruits and/or vegetable matter is extracted using a polar solvent or polar solvent mixture.

3. The plant extract composition of claim 1, wherein the oil soluble constituents have been extracted from edible herbs, spices, fruits and/or vegetables with a relatively non-polar solvent or solvent mixture.

4. The plant extract composition of claim 1, further comprising one or more non-chelating antioxidant components also derived from edible herbs, spices, fruits, vegetables and/or grains.

5. The plant extract composition of claim 1, which is combined with one or more synthetic food grade antioxidants, wherein the combination does not cause an objectionable off-color in a food.

6. The plant extract composition of claim 1, which exhibits anti-microbial and/or antimycotic activity in food compositions into which it is incorporated.

7. The plant extract composition of claim 1, comprising a combination of metal chelating elements derived from previously extracted edible herbs, spices, fruits and/or vegetables.

8. The plant extract composition of claim 1, comprising combinations of metal chelating elements derived from edible herbs, spices, fruits and/or vegetables, further comprising radical scavengers, oxygen scavengers, secondary antioxidants, quenchers and/or antioxidant regenerators derived from natural and/or synthetic sources.

9. A stabilized food, beverage, cosmetic and/or nutritional supplement comprising the plant extract of claim 1 and, optionally, synthetic and/or natural antioxidants of the radical scavenger, oxygen scavenger, secondary antioxidant, quencher and/or antioxidant regenerator types.

10. A method for stabilizing foods, beverages, cosmetics and/or nutritional supplements comprising incorporating an effective amount of the plant extract composition of claim 1 into the food, beverage, cosmetic and or nutritional supplement.

11. The method of claim 10, further comprising incorporating natural and/or synthetic antioxidants into the food, beverage, cosmetic and or nutritional supplement.

12. A method for stabilizing the fresh flavor and preventing the formation of off-flavors in mayonnaise, salad dressings and other oil-in-water emulsion-based food systems comprising incorporating an effective amount of the plant extract composition of claim 1 into the mayonnaise, salad dressings and other oil-in-water emulsion-based food systems.

13. A method for stabilizing the fresh flavor and color and preventing the formation of off-flavors and off-colors in cured meats comprising incorporating an effective amount of the plant extract composition of claim 1 into the cured meats.

14. The method of claim 13, wherein the cured meat is selected from ham, bacon, salt pork, sausage, kippered herring, beef jerky, salami, summer sausage, cold cuts, bologna, pastrami, pepperoni, corned beef, roast beef, hot dogs, dried beef, bratwurst, polish sausage, barbequed pork, pork loin, beef brisket, salmon, liverwurst, pork char sui, prosciutto, culatello, lomo, coppa, bresaola, lardo, guanciale, mocetta, and qadid.

15. A method for stabilizing the fresh flavor and preventing the formation of off-flavors in frying oils and in the foods fried in the oil by treating the frying oil prior to or during the frying operation with an effective amount of the plant extract composition of claim 1.

16. A method for slowing the rate of oxidation, stabilizing the fresh flavor and preventing the formation of off-flavors in extruded human and animal foods comprising incorporating an effective amount of the plant extract composition of claim 1.

17. A method for slowing the rate of oxidation, stabilizing the fresh flavor and preventing the formation of off-flavors in fats and oils comprising polyunsaturated lipids comprising the step of treating the fats and oils with an effective amount of the plant extract composition of claim 1.

18. A method of slowing or preventing the growth of microorganisms in a food composition comprising incorporating the plant extract composition of claim 1 into the food composition in an amount effective to slow or prevent the growth of microorganisms.
